(19) United States
(12) Patent Application Publication
(10) Pub. No.: US 2014/0315258 A1
(43) Pub. Date: Oct. 23, 2014

(54) METHODS FOR CONVERTING CELLULOSIC WASTE TO BIOPRODUCTS

(71) Applicant: ABENGOA BIOENERGY NEW TECHNOLOGIES, LLC, Chesterfield, MO (US)

(72) Inventor: Quang A. Nguyen, Chesterfield, MO (US)

(21) Appl. No.: 14/209,223

(22) Filed: Mar. 13, 2014

Related U.S. Application Data
(60) Provisional application No. 61/783,251, filed on Mar. 14, 2013.

(51) Int. Cl. C12P 19/02; C12P 7/14; C12P 19/14

(52) U.S. Cl. CPC C12P 19/02; C12P 19/14; C12P 7/14
USPC 435/99; 435/162

(57) ABSTRACT

The present invention provides processes for converting cellulosic waste, such as municipal solid waste, to bioproducts such as monosaccharides and fermentation products.

### CROSS REFERENCE TO RELATED APPLICATIONS

This application claims the priority of U.S. Provisional Patent Application Ser. No. 61/783,251, filed Mar. 14, 2013, which is hereby incorporated by reference in its entirety.

### BACKGROUND OF THE INVENTION

The field of the invention generally relates to converting cellulosic waste to bioproducts.

Based on US Environmental Protection Agency data, about 250 million tons of municipal solid waste was generated in the United States in 2010. Solid waste typically comprises organic materials including paper and paperboard, food waste, and yard waste in concentrations of about 29, 14, and 13 percent by weight (“wt. %), respectively. The remainder of the solid waste components typically include about 12 wt. % mixed plastics, about 9 wt. % mixed metals, about 8 wt. % rubber, leather and textiles, about 6 wt. % wood, about 5 wt. % glass, and about 3 wt. % miscellaneous components. In 2010, about one third of the solid waste was recycled and composted, and the remainder was disposed of in landfills.

Solid waste contains high levels of cellulosic fiber (e.g., predominantly cellulose and hemicellulose and, in some waste sources, lesser amounts of ligno-cellulose) and other polysaccharides (e.g., starch) that may be converted to bioproducts such as monosaccharides and fermentation products. More particularly, cellulose and lignocellulose (if present) may be enzymatically hydrolyzed to glucose, and hemicellulose may be enzymatically hydrolyzed to form mixed monosaccharides including xylose, mannose, arabinose and galactose. Problematically, the cellulosic component of solid waste contains high levels contaminants, such as paper fillers, ink and stickies, that reduce the accessibility of enzymes to the cellulosic waste and/or reduce enzyme activity. Of the contaminants, solid waste typically comprises as much as 30 wt. % ash including paper fillers such as calcium carbonate that reduce cellulosic waste bioavailability and kaolin clays that reduce the activity of cellulolytic and hemicellulolytic enzymes. As a result, very high enzyme dosages are required to hydrolyze the cellulosic fibers in solid waste material to fermentable sugars in commercially practical concentrations.

A need therefore exists for improved methods for converting cellulosic waste to bioproducts.

### BRIEF DESCRIPTION OF THE INVENTION

Briefly, the present invention provides process for conversion of cellulosic solid waste to bioproducts.

In one aspect, a process for producing a monosaccharide composition from solid waste is provided. The process comprises forming a solid waste slurry comprising solid waste and water, wherein the slurry comprises from about 2 to about 8 percent by weight total solids, and wherein the solid waste comprises cellulose and ash. The solid waste slurry is pulped to form a pulped solid waste slurry comprising cellulosic fiber. The pulped solid waste slurry is fractionated through a filtration medium having opening of from about 0.2 cm to about 2 cm to form an oversize stream comprising coarse contaminants and an undersize stream comprising cellulosic fibers and light contaminants. The undersize stream is fractionated to form a light contaminant stream comprising ash and a cellulosic fiber stream, wherein the cellulosic fiber stream comprises no more than about 15 percent by ash on a dry basis, and wherein the ash content of the cellulosic fiber stream is less than the ash content of the solid waste on a dry basis. The cellulosic fiber stream is dewatered to form a dewatered cellulosic fiber stream having a solids content of from about 25 wt. % to about 60 wt. % and an aqueous stream. The dewatered cellulosic fiber stream is treated by contact with steam at a treatment temperature of at least 150° C. and a treatment pressure of at least 350 kPa (about 50 psig) to form steam treated cellulosic fiber. A slurry comprising the steam treated cellulosic fiber, water and at least one cellulase enzyme is formed and incubated to form a hydrolyzed cellulosic composition comprising soluble glucose.

### BRIEF DESCRIPTION OF THE DRAWINGS

FIG. 1 depicts a process flow of one aspect of an ethanol process of the present invention.

FIG. 2 depicts a process flow of another aspect of an ethanol process of the present invention.

### DETAILED DESCRIPTION OF THE INVENTION

The process of the present invention is generally directed to the fractionation of solid waste to remove contaminants that interfere with cellulosic fiber bioavailability and the activity of hydrolytic enzymes, and the preparation of a monosaccharide composition from the fractionated cellulosic fibers. The process of the present invention is further directed to the preparation of fermentation products from the monosaccharide composition.

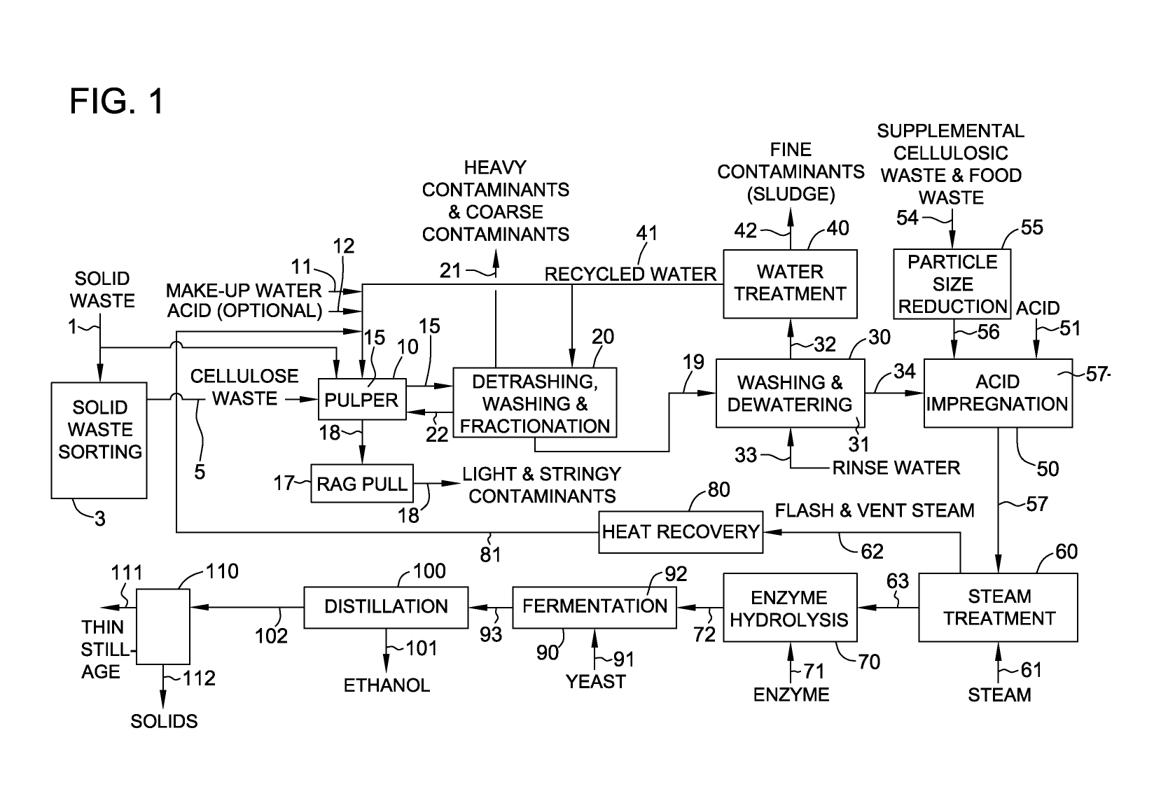

One aspect of the present invention is depicted in FIG. 1. In summary, solid waste 1 (i) may be sorted in a sorting system 3 to form a cellulosic waste stream 5 predominantly comprising paper and paperboard waste that is fed to a pulper 10 and/or (ii) may be fed directly to the pulper 10. In the pulper 10, the cellulosic waste stream 5 and/or the solid waste 1 are combined with make-up water 11 and/or one or more recycle streams such as recycled water 41 or condensed flash and vent steam 81 to form a solid waste slurry 15 comprising cellulosic fibers (e.g., cellulose and hemicellulose) and ash. Optionally, an acid 12 may be added to the pulper 10. As used herein, “ash” is defined as comprising paper and paperboard fillers comprising calcium carbonate and kaolin clays. The solid waste slurry 15 is pulped in the pulper 10 in a pulping operation to form a pulped solid waste slurry, also depicted as 15. The pulped solid waste slurry 15 is processed in a first fractionation (detrashing) step 20 through a first filtration medium to generate a first oversize stream comprising heavy and coarse contaminants 21 and a first undersize stream comprising cellulosic fibers 19. The pulper 10 may further comprise a rag pull system 17 for removing light and stringy contaminants 18. The heavy and coarse contaminants 21 may be washed with recycled water 41 (or process water or other recycle streams that are not depicted in FIG. 1) wherein the wash stream 22 is recycled to the pulper 10. The first undersize stream 19 is then fractionated in a separation device in a second fractionation step, depicted as washing and dewatering 30, to generate a second undersize stream 32 comprising ash particles and cellulosic fines and a second oversize stream 31 comprising cellulosic fibers. The cellulosic fiber stream 31 is washed with rinse water 33 (and/or one or more of process water or recycle streams that are not depicted in FIG. 1). The cellulosic fiber stream 31 is dewatered to form a dewatered cellulosic fiber stream 34. The second undersize stream 32, rinse water 33 and dewatering liquor are forwarded to water treatment 40. Water treatment 40 processes the second undersize stream 32, the rinse water stream 33 and the dewatering liquor to form a fine contaminants sludge stream 42 and a recycled water stream 41 comprising soluble cellulosic fibers, soluble polysaccharides, soluble monosaccharide sugars and cellulosic fines. In some aspects of the present invention depicted in FIG. 1, the cellulosic fiber stream 34 is impregnated with an acid 51 in an acid impregnation system 50 to form an acid impregnated cellulosic biomass 57. Optionally, supplemental cellulosic waste 54, comprising food waste, lawn and garden waste, and/or wood, can be combined with the cellulosic fiber stream 34 in the acid impregnation system 50 to form the acid impregnated biomass 57. The supplemental waste 54 is typically processed in a particle size reduction system 55 to form a milled pre-treated supplemental waste 56 having a particle size distribution suitable for subsequent processing. The acid impregnated cellulosic biomass 57 is transferred to a steam treatment system 60 wherein it is subjected to high temperature and pressure steam 61 followed by expansion by pressure reduction to form a steam treated cellulosic biomass 63. Flash and vent steam 62 are condensed in a heat recovery system 80 to form an acid condensate recycle stream 81 that may be recycled to the pulper 10. The steam treated cellulosic biomass 63 is contacted in an enzyme hydrolysis vessel 70 with an enzyme source 71 comprising at least one cellulase, and optionally at least one additional enzyme (e.g., a hemicellulase), followed by incubation to generate a hydrolyzed cellulosic composition comprising glucose 72. In a fermentation system 90, the hydrolyzed cellulosic composition 72 is combined with a yeast source 91 comprising at least one yeast species capable of converting glucose to ethanol to form a fermentation medium 92, followed by incubation thereof to generate beer 93 comprising ethanol. The beer 93 is distilled in a distillation system 100 to produce ethanol 101 and stillage 102. The stillage 102 may be further processed in a solid-liquid separation device 110 to separate a thin stillage 111 from a solid material 112.

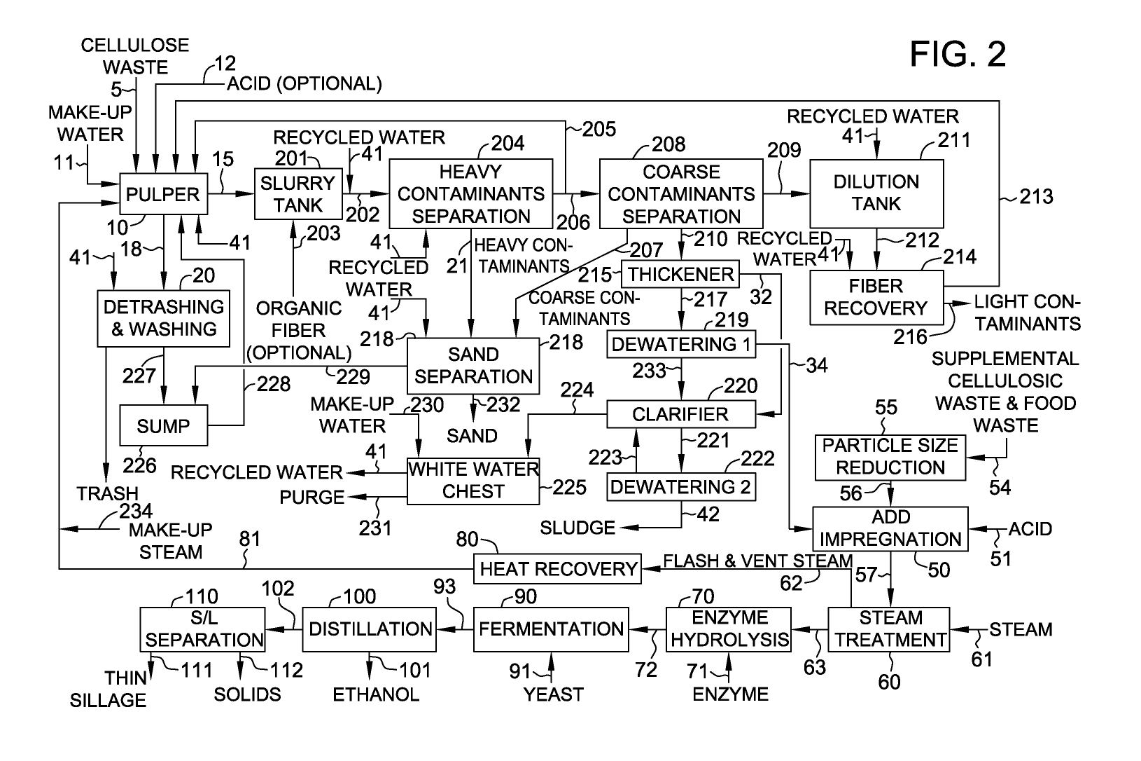

Another aspect of the present invention is depicted in FIG. 2. In summary, a cellulosic waste stream 5 from a municipal solid waste (MSW) sorting facility is conveyed to a pulper 10. This waste stream may be a single stream or multiple streams depending on the MSW sorting configuration. Various aqueous streams including make-up water 11, a filtrate 213 from a fiber recovery unit 214, recycled water 41 from a white water chest 225, wash water 228 from a sump 226, a condensed flash and vent stream 81 from a heat recovery unit 80, and a make-up steam condensate 234 may be added to the pulper 10 to form a slurry. The pulper is equipped with a screen with opening about 6 mm to about 15 mm through which the pulp slurry 15 and smaller contaminants pass and which are forwarded to a slurry tank 201. The oversize material 18 retained on the screen is removed from the pulper 10 by a detrashing and washing unit 20 or manually by briefly shutting down the feed streams to the pulper and flushing the trash from the pulper into a wire mesh screen tote positioned above the sump. In some aspects of the present invention, not depicted in FIG. 2, the detrashing and washing unit 20 is integral with the pulper 10. The detrashing and washing unit 20 washes the oversize material 18 with recycled water 41 to form a trash stream for disposal and detrashing and a washing stream (comprising fiber) 227 that is sent forward to the sump 226. The detrashing and washing stream 227 is combined with a sand separation wash stream 229 in the sump 226 to form a sump stream 228 that is recycled to the pulper 10. A hygienized organic fiber 203 may optionally be combined with the pulp slurry 15 in the slurry tank 201. The pulp slurry is diluted, such as with recycled water 41 to form a slurry tank suspension 202 that is forwarded to heavy contaminants separation 204. In some aspects of the present invention, the hygienized organic fiber 203 comprises steam pretreated fiber not requiring a pulping pretreatment step to break down fiber bundles and dislodge contaminants. In the heavy contaminants separation 204, high density (heavy) contaminants 21 (such as, for example, tramp metal, gravel, staples) are separated, washed with process water 41, and forwarded to the sand separation unit 218. In the sand separation unit, the high density contaminants 21 are washed with process water 41 to form a sand separation wash stream 229 comprising recovered fiber that is forwarded to the sump 226 and a sand waste stream 232 (e.g., tramp metal, gravel, staples). A pulp slurry stream 206 from the heavy contaminants separation 204 is forwarded to a coarse contaminants separation 208. In some aspects of the present invention, at least a portion of the pulp slurry stream 206 may be recycled to the pulper as stream 205. In the coarse contaminants separation 208, the slurry stream 206 is fractionated to form (i) a coarse contaminant stream 207 that is forwarded to sand separation 218, (ii) a light contaminant stream 209 that is forwarded to the dilution tank 211 and (iii) a cleaned fiber slurry 210 that is forwarded to the thickener 215. In the dilution tank 211, the light contaminant stream 209 is diluted with recycled water 41 to reduce the total solids content by from about 25% to about 75% and form a diluted slurry 212 that is forwarded to fiber recovery 214. In fiber recovery 214, the fibers in diluted slurry 212 are washed with recycled water 41 to recover entrained fiber and form a light contaminant stream 216 that is purged from the process and a wash water stream comprising recovered fiber 213 that is recycled to the pulper 10. In the thickener 215, the solids content of the cleaned fiber slurry is concentrated by a factor of from about 200% to about 500% to form a thickened slurry stream 217 and an undersize stream 32 comprising fine contaminants. The undersize stream 32 is forwarded to a clarifier 220 and the thickened slurry stream 217 is forwarded to a dewatering 1 unit 219. In the dewatering 1 unit 219, the thickened slurry stream 217 is dewatered to increase the solids content by from about 200% to about 500% to form a dewatered cellulosic fiber stream 34 that is forwarded to acid impregnation 50 and a liquid stream 233 comprising fines that is forwarded to the clarifier 220. The clarifier 220 separates fines (mainly fiber and paper filler) to form a clarified liquid 224 that is forwarded to the white water chest 225 and a slurry comprising fines 221 that is forwarded to a dewatering 2 unit 222. In the dewatering 2 unit 222, the slurry comprising fines 221 is dewatered to form a fine contaminants stream 42 comprising about 20 to about 40 wt. % total solids and a liquid stream 223 that is recycled to the clarifier 220. In some aspects of the present invention, not depicted in FIG. 2, if the fine contaminants stream 42 comprises a high content of cellulosic fiber, at least a portion of the stream is forwarded to enzyme hydrolysis 70. In some other aspects of the present invention, if the fine contaminants stream 42 comprises a high content of ash, at least a portion of the stream is purged from the process. In the white water chest 225, make-up water 230 is added as necessary to maintain a generally constant layer, sediment 231 is periodically purged from the process and recycled water 41 is distributed to various unit operations as depicted in FIG. 2 and described above.

Acid impregnation, steam treatment, enzyme hydrolysis, fermentation, distillation and solid/liquid separation of the process depicted in FIG. 2 proceeds as generally described above in connection with FIG. 1. In particular, in some aspects of the present invention depicted in FIG. 2, the cellulosic fiber stream 34 is impregnated with an acid 51 in the acid impregnation system 50 to form an acid impregnated cellulosic biomass 57. Optionally, the supplemental cellulosic waste 54, comprising food waste, lawn and garden waste, and/or wood, can be combined with the cellulosic fiber stream 34 in the acid impregnation system 50 to form the acid impregnated biomass 57. The supplemental waste 54 is typically processed in a particle size reduction system 55 to form a milled pre-treated supplemental waste 56 having a particle size distribution suitable for subsequent processing. The acid impregnated cellulosic biomass 57 is transferred to a steam treatment system 60 wherein it is subjected to high temperature and pressure steam 61 followed by expansion by pressure reduction to form a steam treated cellulosic biomass 63. The flash and vent steam 62 is condensed in a heat recovery system 80 to form an acid condensate recycle stream 81 that may be recycled to the pulper 10. The steam treated cellulosic biomass 63 is contacted in an enzyme hydrolysis vessel 70 with an enzyme source 71 comprising at least one cellulase, and optionally at least one additional enzyme (e.g., a hemicellulase), followed by incubation to generate a hydrolyzed cellulosic composition comprising glucose 72. In a fermentation system 90, the hydrolyzed cellulosic composition 72 is combined with a yeast source 91 comprising at least one yeast species capable of converting glucose to ethanol to form a fermentation medium, followed by incubation thereof to generate beer 93 comprising ethanol. The beer 93 is distilled in a distillation system 100 to produce ethanol 101 and stillage 102. The stillage 102 may be further processed in a solid-liquid separation device 110 to separate thin stillage 111 from solid material 112.

In some aspects of the present invention, not depicted in FIGS. 1 and 2, the dewatered cellulosic fiber stream 34 is not subjected to acid treatment prior to steam treatment. In some other aspects of the present invention, not depicted in FIGS. 1 and 2, acid and steam treatment is not done, and the dewatered cellulosic fiber stream 34 is processed in a particle size reduction step.

### Solid Waste

In any of the various aspects of the present invention, sorted and non-sorted solid (cellulosic) waste is collectively termed “solid waste.” Mixed solid waste material, such as municipal solid waste, typically contains a combination of components including food waste, garden/yard waste, paper products, plastic, rubber, textiles, wood, metal products, glass and ceramic products, ash, rocks and dirt. Compositionally, mixed solid waste typically comprises, among other components, (i) cellulosic material comprising cellulose and hemicellulose, and, in some aspects, lignocellulose (generally a component of lawn, garden and wood waster), (ii) paper fillers, (iii) stickies and (iv) oils. Such cellulose-containing solid waste is termed “cellulosic waste”. Paper fillers are compounds that typically are added to paper in concentrations of up to about 50% by weight (“wt. %) to impart softness, flexibility, and optical properties such as opacity and color. Paper fillers may be collectively referred to as ash and comprise inorganic compounds and pigments including clay (e.g., kaolin clay), calcium carbonate and other calcium containing components, ink particles, titanium dioxide, talcum, magnesium-containing components, sodium-containing components, potassium-containing components, phosphorus-containing components and aluminum containing components. The total ash content of solid (cellulosic) waste is typically up to about 30 wt. % on a dry basis. Ash content may be measured by any suitable method known in the art such as a dry ashing procedure wherein the sample is weighed and heated in an oven or furnace in the presence of air to between 500 and 600° C. to vaporize water and other volatile materials and incinerate organic substances to CO₂, H₂O and N₂, and to convert most minerals to non-volatile oxides, sulfates, phosphates, chlorides and silicates. The incinerated sample is weighed and compared to the original sample weight to determine the ash concentration. Generally, the clay and inorganic ash components (e.g., calcium carbonate) have an average particle size in the range of from about 1 micron to about 5 microns. Stickies are generally comprised of polymer aggregates and may include a mixture of, for instance, glues, hot melt plastics, latex coatings and adhesives. Stickies and ink particles typically have an average particle size of from about 1 micron to about 100 microns. Cellulose, hemicellulose and lignocellulose fibers in solid waste is generally sourced from a combination of hardwood, softwood and non-wood plant fibers. Hardwood fibers typically have an average fiber length of from about 0.8 mm to about 1.2 mm and an average diameter (width) of from about 15 microns to about 30 microns. Softwood fibers typically have an average fiber length of from about 3 mm to about 7 mm and an average width of from about 30 to about 60 microns. Non-wood plants typically have an average fiber length of from about 1 to about 3 mm and an average width of from about 10 microns to about 30 microns. The ratio of hardwood, softwood and non-wood plant fibers in the cellulosic fibers various with the source and composition of the solid (cellulosic) waste.

In some aspects of the present invention, non-sorted solid (cellulosic) waste may be added directly to the pulper. In some other aspects of the present invention, recovered paper and paperboard waste streams generated in commercial waste sorting operations may be added to the pulper. In some other aspects of the present invention, a mixture of solid waste and recovered pulp/paperboard may be added to the pulper. In some further aspects of the present invention, supplemental cellulosic waste, such as solid food waste, lawn and garden waste, tree pruning, agricultural residues (e.g., straw, corn stover, husks), grasses, and/or wood may be added to the process in a post-pulping step, such as in an acid impregnation and/or steam treatment step. Lawn, garden and wood waste is generally the source of the majority of the lignocellulose component of the cellulosic waste.

### Solid Waste Slurry and Pulping

Referring to FIGS. 1 and 2, the solid waste material 1 and/or 5 is combined with the make-up water 11 and/or one or more recycle streams such as the recycle water stream 41 or the condensed flash and vent steam 81 in the pulper 10 to form a solid waste slurry 15 comprising cellulosic fibers and ash from which a pulped solid waste slurry 15 is formed. The pulping action, or shear, is generally sufficient to break up large paper and cardboard pieces, defiber cellulosic material such as by separation of the strands of cellulosic fibers. The pulping action loosens, separates and liberates contaminants such as dirt, rocks, gravel, metal, plastic, staples, glass, sand, textiles (e.g., “rags”) and the entrapped filler particles (e.g., ash) into the slurry as a suspended material and particulate and/or dissolved components. Preservation of fiber length is not narrowly critical to achieve the objects of the present invention. The present process therefore differs from conventional paper recycling pulping in this respect wherein high consistency pulping is used for the general preservation of fiber length.

The solid waste slurry may be formed in any suitable mixing vessel. In some aspects of the present invention, the slurry is formed in a pulper. Suitable pulpers are known in the art and include batch and continuous pulpers manufactured by, for instance, Andritz and Voith Paper. In some further aspects of the present invention, the pulpers are detrashing pulpers that are capable of extracting large contaminants such a rocks, gravel, plastic pieces, metal pieces, rags, and strings. In some aspects of the present invention, the pulper optionally comprises a detrashing unit and a rag extraction units. The detrashing unit generally removes heavy contaminants (e.g., metal, rock, gravel and glass) and the rag extractor generally removes cloth, strings and plastic. The heavy contaminants may be washed with one or more recycle streams to recover cellulosic fibers, and the wash stream may suitably be reintroduced into the process in the pulping operation.

To achieve the objective of releasing fillers from the fibers, low to medium consistency pulping is generally preferred over high consistency pulping. In any of the various aspects of the present invention, the insoluble solid content of the pulping slurry is about 2 wt. %, about 3 wt. %, about 4 wt. %, about 5 wt. %, about 6 wt. %, about 7 wt. % or about 8 wt. % total solids (“TS”), and ranges thereof, such as from about 2 wt. % to about 8 wt. %, about 2 wt. % to about 7 wt. %, about 2 wt. % to about 6 wt. %, about 2 wt. % to about 5 wt. %, about 3 wt. % to about 8 wt. %, about 4 wt. % to about 8 wt. %, about 5 wt. % to about 8 wt. %, about 4 wt. % to about 7 wt. %, about 4 wt. % to about 6 wt. %, or from about 4 wt. % to about 5 wt. % TS. The energy input to the pulper is about 5 kW/m³, about 6 kW/m³, about 7 kW/m³ about 8 kW/m³, or about 9 kW/m³, and ranges thereof. In some aspects of the present invention, the average fiber length of the cellulosic fibers in a pulped slurry is about 95%, about 90%, about 85%, about 80%, about 75%, about 70%, about 65%, about 60%, about 55%, or about 50%, and ranges thereof, of the average fiber length of a plurality of cellulosic fibers in the solid waste, such as from about 50% to about 95%, from about 60% to about 90%, or from about 70% to about 90%. The temperature of pulping (solid waste) slurry is adjusted to at least about 30° C., such as about 40° C., about 50° C., about 60° C., about 70° C., about 75° C., about 80° C., or more, and ranges thereof, such as from about 50° C. to about 80° C. from about 40° C. to about 70° C., or from about 60° C. to about 80° C. In some aspects of the present invention, make-up steam is utilized as the heat source. The average residence time in the pulper is typically from about 4 minutes to about 10 minutes, but may suitably vary depending on the characteristics of the waste and the consistency of the slurry in the pulper.

In some aspects of the present invention, the pH of the solid waste slurry is adjusted to about 3, about 3.5, about 4, about 4.5, about 5, about 5.5 or about 6.5, and ranges thereof, such as from about 3 to about 6 or from about 4 to about 5 by the addition of acid. As depicted in FIGS. 1 and 2, an acid may be added to the pulper from an acid source 12 and/or from one or more recycle streams such as the condensed flash and vent steam 81 that may comprise acid. Acidic pH serves to dissolve ash components such as calcium carbonate and to inhibit microbial growth. Any suitable acid, such as a mineral acid, for instance, sulfuric acid, hydrochloric acid or nitric acid, may be used for acidic pH adjustment and any suitable base such as ammonium hydroxide or sodium hydroxide may be used for basic pH adjustment.

In some other aspects of the present invention, a solid slurry pH of from about 4 to about 8 used, such as about 4, about 5, about 6, about 7 or about 8.

The pulping action separates large contaminants such as rocks, metal, glass, plastics and textiles (e.g., “rags”) from the pulped solid waste slurry. In some aspects of the present invention, the pulper comprises a detrashing unit and/or a rag extraction unit. Thus, the rag pull unit 17 and/or the detrashing unit 20 as depicted on FIGS. 1 and 2 could be integral to the pulper 10. In some aspects of the present invention, the screen opening for the pulper is typically about 0.5 cm, about 1 cm or about 1.5 cm, and ranges thereof, such as from about 0.5 cm to about 1.5 cm. The screen opening for the detrashing unit is typically about 1 cm, about 1.5 cm, or about 2.5 cm, and ranges thereof, such as from about 1.5 cm to about 2.5 cm. The filtration medium opening size may be suitably selected based on the particle size distribution of the pulped solid waste slurry or diluted slurry. For instance, large particle sizes generally require larger screen openings in the pulper detrashing unit. The detrashing unit generally removes heavy contaminants (e.g., metal, rock, gravel and glass) and the rag extractor generally removes cloth, strings and plastic.

In some aspects of the present invention depicted in FIG. 2, the de-trashed solid waste slurry 15 is forwarded to the slurry tank 201. Generally, the slurry tank functions as a surge tank for down-stream unit operations thereby allowing the pulper to be taken out of service. For instance, the pulper may be taken out of service periodically for maintenance, inspection and/or for manual removal of oversize material (for instance, by flushing) such as metal, plastic or glass. In some aspects of the present invention, organic fiber not requiring pulping can be added to the slurry tank. For instance, a hygienated organic fiber stream from acid impregnation, thin stillage and/or solids from stillage separation can be combined with de-trashed solid waste slurry in the slurry tank. The slurry tank 201 is suitably an agitated vessel such as a vertical cylindrical tank with a multi-impeller agitator.

### Fractionation

In further reference to FIG. 1, the pulped solid waste 15 is fractionated to form a heavy/coarse contaminant stream 21 and a cellulosic fiber stream 19. In some aspects of the present invention, prior to fractionation, the pulped solid waste slurry may be diluted to about 1 wt. %, about 1.5 wt. %, about 2 wt. %, about 3 wt. % or about 4 wt. % TS, and ranges thereof, such as from about 1 wt. % to about 4 wt. %, from about 1.5 wt. % to about 4 wt. %, or from about 1.5 wt. % to about 2.5 wt. % with make-up water, recycle streams comprising water and/or steam condensate to form a diluted slurry. In some other aspects, the pH of the diluted slurry may be adjusted to about 6, about 5, about 4, about 3, about 2, and ranges thereof, such as from about 2 to about 6, from about 3 to about 6, or from about 4 to about 6. An acidic pH is believed to increase the solubility of calcium carbonate and other inorganic ash components. The temperature of the diluted slurry is typically maintained above about 30° C.

In some aspects of the present invention, the pulped solid waste slurry or the diluted slurry may be passed through a filtration medium having an opening of about 0.5 cm, about 1 cm, about 1.5 cm or about 2 cm, and ranges thereof, such as from about 0.5 cm to about 2 cm to form a first oversize stream comprising heavy and coarse contaminants and a first undersize stream comprising cellulosic fibers. The filtration medium opening size may be suitably selected based on the particle size distribution of the pulped solid waste slurry or diluted slurry.

The heavy and coarse contaminants are washed with any one of water, recycled wash water, flash and vent steam condensate, thin stillage and combinations thereof, to form a wash stream 22 comprising cellulosic fibers and a washed heavy/coarse contaminant stream 21. The wash stream is recycled to the pulper for cellulose recovery. The washed heavy/coarse contaminants may be discarded as waste or further processed for the recovery of components such as metals and plastics.

In further reference to FIG. 1, The first undersize stream comprising cellulosic fibers 19 is fractionated and washed in a fractionating/washing/dewatering device 30 to form a second undersize stream 32 predominantly comprising fine contaminants (e.g., loose kaolin clay, calcium carbonate, ink particles and stickies) and a second oversize stream 31 predominantly comprising cellulosic fibers. As used herein, “predominantly” means greater than 50%, at least 75%, at least 90% or at least 95% on a population%, w/w%, w/v% or v/v% basis, based on TS. Examples of fractionation and dewatering devices suitable for practice of any of the various aspects of the present invention such as depicted in FIGS. 1 and 2 include, without limitation, a vibratory screen, a wire/belt washer, a rotary screen, a paddle screen, a pressure filter, a pressure screen, a centrifugal sifter and a hydrocyclone. Suitable separation media include slotted bars, perforated plates, perforated sheets, and screens. The fractionation and washing device may be single stage or multiple stage, and batch or continuous. In any of the various fractionation device aspects of the present inventions, the separation media has an opening of about 0.2 mm, about 0.5 mm, about 1 mm, about 1.5 mm, about 2 mm, about 2.5 mm, about 3 mm, about 3.5 mm, about 4 mm, about 4.5 mm or about 5 mm, and ranges thereof, such as from about 0.2 to about 5 mm, from about 1 to about 5 mm, from about 2 to about 5 mm, or from about 0.2 to about 2 mm. The second undersize stream is characterized as an average particle size range of from about 1 to about 100 microns. More particularly, a plurality of the ash particles contained in the second undersize stream have an average particle size of about 1 micron, about 5, about 10, about 20, about 30, about 40, about 50, about 60, about 70, about 80, about 90, or about 100 microns, and ranges thereof, such as from about 1 to about 100 microns, from about 20 to about 100 microns, from about 50 to about 100 microns, from about 1 to about 50 microns, or from about 20 to about 50 microns.

As shown in FIG. 1, the second oversize stream containing cellulosic fiber 31 is retained on the filtration media 30 and washed with water 33. The cellulosic fiber may optionally or additionally be washed with recycled wash water 41, flash and vent steam condensate 81, thin stillage 111 and combinations thereof. The wash stream is combined with the second undersize stream 32, to form a wash stream comprising fine contaminants. The ash content of the washed cellulosic fiber 31 is less than the ash content of the solid waste on a TS or dry basis. More particularly, the washed second oversize stream comprises less than about 15 wt. %, less that about 14 wt. %, less that about 13 wt. %, less that about 12 wt. %, less that about 11 wt. %, less that about 10 wt. %, less that about 9 wt. %, less that about 8 wt. %, less that about 7 wt. %, less that about 6 wt. % or less than about 5 wt. % ash on a total solids basis. In some aspects of the present invention, the washed second oversize stream comprises from about 5 wt. % to about 15 wt. %, from about 10 wt. % to about 15 wt. %, or from about 5 wt. % to about 10 wt. % ash on a TS or dry basis. The washed cellulosic fiber stream is characterized as having an average fiber length of from about 1 mm to about 4 mm and an average fiber width of from about 10 to about 50 microns. More particularly, a plurality of cellulosic fibers contained therein have (i) an average fiber length of about 1 mm, about 1.5 mm, about 2 mm, about 2.5 mm, about 3 mm, about 3.5 mm or about 4 mm, and ranges thereof, such as from about 1 mm to about 4 mm, from about 1 mm to about 3 mm, or from about 2 mm to about 4 mm and (ii) an average fiber width of about 10 microns, about 15 microns, about 20 microns, about 25 microns, about 30 microns, about 35 microns, about 40 microns, about 45 microns or about 50 microns, and ranges thereof, such as from about 10 microns to about 50 microns, from about 25 microns to about 50 microns or from about 10 microns to about 25 microns. In some aspects of the present invention, a plurality of the cellulosic fibers are about 95%, about 90%, about 85%, about 80%, about 75%, about 70%, about 65%, about 60%, about 55% or about 50%, and ranges thereof, of the average length of a plurality of cellulosic fibers contained in the solid waste, such as from about 50% to about 95%, from about 75% to about 95%, or from about 50% to about 75%.

The washed cellulosic fiber stream is optionally dewatered to produce a second oversize liquid stream and a dewatered cellulosic fiber stream 34 having a TS content of about 15%, about 20%, about 25%, about 30%, about 35%, about 40%, about 45%, about 50%, about 55% or about 60%, and ranges thereof, such as from about 15% to about 60%, about 30% to about 60%, about 40% to about 60%, from about 20% to about 40%, or from about 35% to about 45%. The dewatering equipment may be suitably selected from a centrifuge, filter press, screw press, belt press, belt filter or roll press. The liquid stream may be recycled to the pulper and/or used to wash the first undersize stream. In some aspects, at least about 25%, at least about 50%, at least about 75% or at least about 90% of the second oversize liquid stream is recycled to the pulper.

In aspects of the invention depicted in FIG. 2, de-trashed solid waste slurry 15 is diluted from a concentration of from about 4 wt. % to about 8 wt. % total solids with at least one of make-up water (not depicted), recycled water, or an aqueous recycle stream (not depicted) in slurry tank 201 or in the suction side of slurry tank 201 discharge pump to form diluted slurry tank suspension 202 having a total solids concentration of about 2 wt. %, about 3 wt. %, about 4 wt. % or about 5 wt. %, and ranges thereof, such as from about 2 wt. % to about 5 wt. %, from about 2 wt. % to about 4 wt. % or from about 2 wt. % to about 3 wt. %.

As further depicted in FIG. 2, the diluted slurry tank suspension 202 is processed in a heavy contaminants separation 204 operation wherein heavy contaminants (such as gravel, metal and staples) are separated and removed from the diluted slurry tank suspension to form a heavy contaminants stream 21 that is forwarded to the sand separation 218 and the pulp slurry stream 206 that is forwarded to the coarse contaminant separation 208. The heavy contaminant may be washed with process make-up water (not depicted), recycled water 41 or any of the various aqueous processing streams of the present invention (not depicted) wherein the wash stream predominantly passes forward with pulp slurry stream. At least a portion of the pulp slurry stream may be recycled to the pulper as stream 205. Suitable equipment for heavy contaminants separation includes any of the various high density cleaners known in the art such as hydrocyclones.

As further depicted in FIG. 2, the pulp slurry stream 206 is processed in a coarse contamination separation step wherein coarse contaminants having a particles size of greater than about 3 mm are separated and form the coarse contaminant stream 207 that is forwarded to the sand separation 218 operation. The cleaned fiber slurry stream 210 and the light contaminant stream 209 are also formed. Suitable equipment for coarse contaminant separation includes any of the various screening and filtering methods known in the art such as vibrating screens, gravity feed boxes, pressure feed boxes, belt filter press, drum screens and pressure screens. Suitable screens include single and multi-stage screens. In some aspects of the present invention two or more separators may be operated in series. For instance, a first (primary) screen having openings of about 3 mm may generate an oversize stream comprising coarse contaminant stream 207, a light contaminant stream 209 and an undersize stream comprising cleaned fiber slurry stream 210. An example of a suitable apparatus includes a rotating vertical pressure screen. Coarse and light contaminant stream may then be passed through a second screen having openings of about 6 mm to generate the coarse contaminant stream 207 and the light contaminant stream 209. An example of a suitable apparatus includes a vibrating screen. In any of the various separation aspects of the present invention, those skilled in the art may suitably select screen sizes, equipment type and associated arrangements thereof based on the characteristics of the pulp slurry stream 206 in order to achieve the objects of the present invention.

As further depicted in FIG. 2, heavy contaminants and coarse contaminants (solid) are separated and isolated from organic fibers (slurry) in the sand separation 218 in a solid-liquid separation process. The heavy contaminants and coarse contaminants are washed with any of process make-up water (not depicted), recycled water 41 or any of the various aqueous processing streams of the present invention (not depicted) to recover entrained organic fibers. The slurry and wash streams are combined to form the sand separation wash stream 229 that is forwarded to the sump 226. Washed heavy contaminants are typically purged from the process. Suitable equipment for sand separation includes an inclined screw conveyor with washing capability and various screening and filtering methods known in the art such as vibrating screens. Those skilled in the art may suitably select screen sizes, equipment type and associated arrangements thereof based on the characteristics of the combined streams in the sand separation step in order to achieve the objects of the present invention.

As further depicted in FIG. 2, organic fiber is recovered from the light contaminant stream 209. The light contaminant stream is combined with any of process make-up water (not depicted), recycled water 41 or any of the various aqueous processing streams of the present invention (not depicted) in a dilution tank to dilute the total solid content of from about 2 wt. % to about 5 wt. % to less than about 2 wt. %, such as between about 1 wt. % and about 2 wt. % or between about 1.5 wt. % and about 2 wt. %. Suitable dilution tank designs are known to those skilled in the art and include an agitated vessel such as a vertical cylindrical tank with a multi-impeller agitator. The diluted slurry 212 is forwarded to fiber recovery 214 for separation and isolation of light contaminants (solid) from organic fibers (slurry). The light contaminants are washed with any of process make-up water, recycled water 41 or any of the various aqueous processing streams of the present invention to recover entrained organic fiber and generate the wash water stream comprising recovered fiber 213 that is recycled to the pulper 10. The washed light contaminants are purged from the process. Suitable equipment for fiber recovery includes a vibrating screen, rotary screen, static screen (such as a side hill screen), and a slotted screw conveyor. Those skilled in the art may suitably select screen sizes, equipment type and associated arrangements thereof based on the characteristics of the diluted slurry in order to achieve the objects of the present invention.

As further depicted in FIG. 2, the cleaned fiber slurry stream 210 is concentrated in a thickening step to increase the solids content of the cleaned fiber slurry by a factor of from about 150% to about 600% and form the thickened slurry stream 217 and the undersize stream 32 comprising fine contaminants. The undersize stream 32 is forwarded to the clarifier 220 and the thickened slurry stream 217 is forwarded to the dewatering 1 unit 219. The total solids content in the thickened slurry stream is increased from about 2 wt. % to about 3 wt. % to from about 3 wt. % to about 12 wt. %, or from about 8 wt. % to about 12 wt. %, such as about 10 wt. %. Suitable thickening equipment includes inclined screw conveyors, slotted screw conveyors and centrifuges. For instance, in some aspects of the present invention, a Hydradenser available from Kadant Black Clawson could be used. Those skilled in the art may suitably select equipment type, equipment specifications and associated arrangements thereof based on the characteristics of the cleaned fiber slurry stream in order to achieve the objects of the present invention.

As further depicted in FIG. 2, the thickened pulp slurry from the thickener 217 is processed in the first dewatering step to form the dewatered cellulosic fiber stream 34 that is forwarded to acid impregnation 50 and the liquid stream 233 comprising fines that is forwarded to the clarifier 220. Suitable dewatering equipment includes a screw press, however, those skilled in the art may suitably select dewatering equipment based on the characteristics of the thickened slurry in order to achieve the objects of the present invention. The total solids content of the dewatered cellulosic fiber stream is about 25 wt. %, about 30 wt. %, about 35 wt. %, about 40 wt. %, about 45 wt. %, about 50 wt. %, about 55 wt. %, or about 60 wt. %, and ranges thereof, such as from about 25 wt. % to about 60 wt. %, from about 25 wt. % to about 40 wt. %, or from about 35 wt. % to about 40 wt. %.

As further depicted in FIG. 2, the liquid streams 233 and 223 from the first and second dewatering steps, respectively, and the undersize stream 32 from the thickener are processed to separate fine particles (such as paper filler and fiber fines) to generate the sludge stream 221 and the clarified liquid stream 224 that is forwarded to the white water chest 225. The clarified liquid stream is characterized by a total solids content of less than about 1 wt. % and the sludge stream is characterized by a total solids content of about 3 wt. %. Suitable clarification equipment includes dissolved air flotation (such as described below for water treatment), mechanical settling clarification, inclined surface solids contact, horizontal flow apparatuses, and vertical flow solids contact. Those skilled in the art may suitably select equipment type, equipment specifications and associated arrangements thereof based on the characteristics of the combined input streams 32, 223 and 233 in order to achieve the objects of the present invention. The white water chest generally provides surge capacity for recycled water and make-up process water, and delivers recycled water to various unit operations described herein. In typical operation, make-up water is added to maintain a constant level. Sediment collected in the white water chest is periodically purged out of the process as stream 231.

As depicted in FIG. 2, the sludge stream 221 is dewatered in the second dewatering step 222 to form the sludge stream 42 and the liquid stream 233. The total solids content of the sludge stream is increased from about 3 wt. % to about 20 wt. %, about 25 wt. %, about 30 wt. %, about 35 wt. % or about 40 wt. %, and ranges thereof, such as from about 20 wt. % to about 40 wt. % or from about 25 wt. % to about 35 wt. %. Suitable dewatering equipment includes a belt press, however, those skilled in the art may suitably select dewatering equipment based on the characteristics of the sludge stream in order to achieve the objects of the present invention. The liquid stream is recycled to the clarifier. In some aspect of the present invention, and the sludge stream is purged. The fine contaminant stream 42 may be analyzed for cellulosic fiber content and ash by methods known in the art. If the stream contains sufficiently high cellulosic fiber and low contaminant content, at least a portion of the stream may be forwarded to enzymatic hydrolysis for the generation of sugars. If the stream does not contain sufficiently high cellulosic fiber and low contaminant content, the stream may be purged from the process. Any recycle decision may be based on the nature of the recovery process being practiced and is within the purview of the skilled artisan.

### Water Treatment

In some aspects of the present invention, one or more process streams described herein, such as a cellulosic fiber wash stream, may be processed in a water treatment operation to form (i) a recycle stream comprising water, soluble polysaccharides, monosaccharides, insoluble pulp fines, soluble ash and insoluble ash fines, such as the recycled water stream 41 depicted in FIGS. 1 and 2, and (ii) a fine contaminants stream. Conventional water treatment devices such as, for instance, clarifiers and hydrocyclones can be used to separate the heavier fine contaminants from the cellulosic fines. The recycle stream may be suitably reintroduced in the process in the pulping operation or utilized as wash water, such as for instance, for the washing either the first oversize fraction or the fraction generated in the detrashing operation. The fine contaminant stream is generally purged from the process in the form of a sludge, slurry or filter cake. In some aspects of the present invention, the recycle stream and/or fine contaminants stream may be dewatered using solid-liquid separation equipment known in the art such as, for instance, a screw press, belt press or filter press.

In some aspects of the present invention the wash stream is processed using added coagulants or flocculants and/or by air flotation may to facilitate the separation of fiber fines from the paper filler. Generally, coagulants neutralize the repulsive electrical charges (typically negative) surrounding particles allowing them to aggregate. Flocculants facilitate the agglomeration or aggregation of the coagulated particles to form larger floccules and thereby hasten gravitational settling. Some coagulants serve a dual purpose of both coagulation and flocculation in that they create large floccules that readily settle. Suitable coagulants are known in the art and include those based on aluminum and those based on iron. Aluminum coagulants include aluminum sulfate, aluminum chloride and sodium aluminate. Iron coagulants include ferric sulfate, ferrous sulfate, ferric chloride and ferric chloride sulfate. Other chemicals used as coagulants include hydrated lime and magnesium carbonate. Suitable flocculants include cationic or anionic polymers and copolymers having medium or high charge, such as including, but not limited to, acrylamide copolymers or polyacrylamide polymers. In air flotation, also termed dissolved air flotation (“DAF”), air is dissolved in the wash stream under pressure and then released at atmospheric pressure in a float tank or basin. The released air forms bubbles which adhere to the suspended particulate matter causing it to flow to the surface where it may then be removed by skimming.

The fine contaminant stream comprises greater than 50%, 55%, 60%, 65%, 70%, 75%, 80%, 85% or 90% of the fine insoluble contaminants contained in the wash stream from which it was generated. The fine contaminant stream is characterized as comprising calcium carbonate and clay particulate wherein the average size of a plurality of the calcium carbonate and clay particles is about 1 micron, about 2, about 3, about 4, about 5, about 6, about 7, about 8, about 9 or about 10 microns, and ranges thereof, such as from about 1 micron to about 10 microns, from about 5 microns to about 10 microns or from about 1 micron to about 5 microns. The fine contaminant stream is further characterized as comprising ink particulate and stickies wherein the average size of a plurality of the ink particulate and stickies is about 1, about 5, about 10, about 25, about 50, about 75, about 100, about 125, about 150, about 175 or about 200 microns, and ranges thereof, such as from about 1 micron to about 200 microns, from about 1 microns to about 100 microns or from about 100 micron to about 200 microns.

### Post-Treatment

In accordance with the present invention, the dewatered cellulosic fiber stream is processed to improve the enzyme digestibility. Post-treatment may comprise fine milling, acid treatment, steam treatment, and combinations thereof. For instance, and without limitation, post treatment may comprise (i) fine milling, (ii) acid treatment, (iii) steam treatment, or (iv) acid treatment and steam treatment.

In some aspects of the present invention, the cellulosic fiber stream is processed by milling to reduce the particle size. The cellulosic fiber stream may be either dewatered or a stream that has not been dewatered. Any suitable milling device can be used, such as a grinder, hammer mill, crusher, knife mill, chopper, disc mill, a centrifugal mill or a homogenizer. In any of the various milling aspects of the present invention, the particle size of a plurality of milled cellulosic fibers are characterized as having an average fiber length of less than about 500 microns, less than about 400 microns, less than about 300 microns, less than about 200 microns, or less than about 100 microns.

In some other aspects of the present invention, as depicted in FIGS. 1 and 2, the milled or unmilled washed cellulosic fiber 34, collectively referred to as cellulosic biomass, may be treated to provide for increased yield of fermentable sugars. Treatment methods include acid treatment and/or steam treatment at elevated temperature and pressure.

Acid treatment includes addition of acid 51 to the cellulosic biomass 34 in suitable acid impregnation equipment 50 (such as by solid phase mixing in a batch mixer or acid impregnation as a slurry in a vessel) to generate an acid impregnated cellulosic biomass 57. In some acid treatment aspects, the cellulosic biomass is combined with a relatively dilute acid. The primary purpose of acid treatment (referred to herein as acid impregnation or treatment) is to prepare cellulosic biomass for subsequent enzymatic hydrolysis for the production of monosaccharide sugars, such as, for instance, glucose and xylose. Forming acid impregnated cellulosic biomass from the cellulosic biomass can be suitably done by admixing dewatered second oversize steam with acid in a solid phase mixing device such as a ribbon blender or pug mill at a total solids content of from about 30% to about 50% or by soaking in an acid impregnation vessel as a slurry having a total solids content of about 25%, about 20%, about 15%, about 10% or less than about 10%, such as from about 5% to about 25%, from about 5% to about 20% or from about 5% to about 15%.

Acid impregnation by solid phase mixing comprises combining cellulosic biomass having a TS content in excess of about 25 wt. %, such as from about 30 wt. % to about 60 wt. %, with a diluted acid in a batch mixer to form an acid impregnated biomass. The precise configuration of the batch mixer is not narrowly critical and may be readily selected from any suitable apparatus known in the art. For example, acid impregnation as detailed herein may be conducted in a batch mixer (e.g., a pug mixer, paddle mixer or ribbon mixer) or in a continuous mixer (e.g., a pug mixer, paddle mixer, ribbon mixer or mixing screw). In some aspects of the present invention the acid is sprayed onto the cellulosic biomass as it passes through the mixer. In some further aspects, a plurality of spray nozzles are positioned in a first section of the mixer, such as the first half or first third of the mixer. In some other aspects, at least a portion of the acid is sprayed onto the cellulosic biomass before feeding into the mixer. Typically the acid comprises a mineral acid selected from the group consisting of hydrochloric acid, sulfuric acid, sulfurous acid, nitric acid, and combinations thereof. As noted, the primary purpose of acid impregnation is preparation of the cellulosic feedstock for enzymatic hydrolysis to produce fermentable sugars. Accordingly, the acid is typically in the form of a relatively dilute acid. The acid may suitably have a concentration of about 0.5 wt. %, about 1 wt. %, about 1.5 wt. %, about 2 wt. %, about 2.5 wt. %, about 3 wt. %, about 3.5 wt. % or about 4 wt. %, and ranges thereof, such as from about 0.5 wt. % to about 4 wt. %, from about 1 wt. % to about 4 wt. %, or from about 1 wt. % to about 3 wt. %. The acid impregnated cellulosic biomass prepared by solid phase mixing has a total solids content of about 20 wt. %, about 25 wt. %, about 30 wt. %, about 35 wt. %, about 40 wt. %, about 45 wt. %, about 50 wt. %, or about 55 wt. %, and ranges thereof, such as from about 20 wt. % to about 55 wt. %, from about 25 wt. % to about 50 wt. %, from about 30 wt. % to about 50 wt. %, from about 30 wt. % to about 45 wt. %, or from about 35 wt. % to about 45 wt. %. Regardless of the precise composition of the acidic acid, the acid impregnated material has an acid concentration of about 0.01, about 0.02, about 0.03, about 0.04 or about 0.05 kg acid per kg of cellulosic biomass on a TS or dry weight basis. The temperature of the acid introduced into the mixer is generally at least about 30° C., at least about 40° C., or at least about 50° C., such as from about 20° C. to about 95° C., from about 30° C. to about 85° C., or from about 40° C. to about 75° C. The pH of the acid-impregnated cellulosic biomass is preferably less than about 4, less than about 3, or less than about 2, such as about 1. The mixing time for contact the cellulosic biomass and acid is typically from about 1 minute, 5 minutes, 10 minutes, or about 15 minutes, and ranges thereof, such as from about 1 minute to about 15 minutes, from about 2 minutes to about 10 minutes, or from about 3 minutes to about 6 minutes. Prior to further processing, the acid impregnated biomass prepared in the mixer is typically held in an insulated or heat jacketed bin at approximately the mixing temperature for about 5 minutes, about 10 minutes, about 20 minutes, about 30 minutes, about 40 minutes, about 50 minutes, or about 60 minutes, and ranges thereof, such as from about 5 minutes to about 60 minutes, from about 10 minutes to about 60 minutes, from about 10 minutes to about 45 minutes, or from about 15 minutes to about 30 minutes in order to provide a predominantly homogeneous admixture. The temperature of the acid impregnated biomass in the hold tank is typically about 40° C., about 50° C., about 60° C., about 65° C., about 70° C. or about 75° C., and ranges thereof, such as from about 40° C. to about 75° C., or from about 50° C. to about 70° C.

Acid impregnation by soaking generally comprises combining cellulosic biomass with a diluted acid in a vessel with mixing to form a slurry. The precise configuration of the acid impregnation vessel is not narrowly critical and may be readily selected from any suitable apparatus known in the art. For example, acid impregnation as detailed herein may be conducted in a batch reactor (e.g., a stirred-tank reactor) or in a vessel suitable for continuous operation (e.g., a continuous stirred-tank reactor or plug flow reactor). Typically, the acid comprises a mineral acid selected from the group consisting of hydrochloric acid, sulfuric acid, sulfurous acid, nitric acid, and combinations thereof. As noted, the primary purpose of acid impregnation is preparation of the cellulosic biomass for enzymatic hydrolysis to produce fermentable sugars. Accordingly, the acid is typically in the form of a relatively dilute acid. The acid may suitably have a concentration of about 1 wt. %, about 2 wt. %, about 3 wt. %, about 4 wt. %, about 5 wt. %, about 10 wt. % or about 15 wt. %, and ranges thereof, such as from about 0.5 to about 15 wt. %, from about 1 to about 10 wt. %, from about 1 wt. % to about 5 wt. %, or from about 1 wt. % to about 3 wt. %. The slurry has a TS content of about 3 wt. %, about 4 wt. %, about 5 wt. %, about 6 wt. % or about 7 wt. %, and ranges thereof, such as from about 3 wt. % to about 7 wt. %, from about wt. % 3 to about 5 wt. %, from about 4 wt. % to about 6 wt. %, or from about 5 wt. % to about 7 wt. %. Regardless of the precise composition of the acidic acid, the acid impregnated material has an acid loading of about 0.01, about 0.05, about 0.1, about 0.15, about 0.20, about 0.25, about 0.3 or about 0.35 kg acid kg cellulosic biomass on a TS or dry weight basis. The temperature of the acid introduced into acid impregnation vessel is generally at least about 30° C., at least about 40° C., or at least about 50° C., such as from about 20° C. to about 95° C., from about 30° C. to about 85° C., or from about 40° C. to about 75° C. The pH of the acid-impregnated cellulosic biomass is preferably less than about 4, less than about 3, or less than about 2, such as about 1. The contact time for contact of the cellulosic biomass is typically about 1 minute, 5 minutes, 10 minutes, or about 15 minutes, and ranges thereof, such as from about 1 minute to about 15 minutes, from about 2 minutes to about 10 minutes, or from about 3 minutes to about 6 minutes.

The acid soaking step is optionally followed by a draining and dewatering step to remove excess acid solution and to provide a dewatered acid-impregnated cellulosic biomass. Dewatering equipment may be suitably selected from a centrifuge, filter press, screw press, belt press, belt filter or roll press. The solids content of the dewatered acid-impregnated cellulosic biomass is typically about 20 wt. %, about 25 wt. %, about 30 wt. %, about 35 wt. %, about 40 wt. %, about 45 wt. %, about 50 wt. %, about 55 wt. %, about 60 wt. %, about 65 wt. %, or about 70 wt. %, and ranges thereof, such as from about 30 wt. % to about 70 wt. %, from about 35 wt. % to about 65 wt. %, from about 35 wt. % to about 60 wt. %, or from about 35 wt. % to about 50 wt. %. In some dewatering aspects of the present invention, a multi-stage dewatering scheme is employed, such as by dewatering the acid impregnated cellulosic biomass to a TS of about 15 wt. %, about 20 wt. %, about 25 wt. %, or about 30 wt. %, and ranges thereof, such as from about 15 to about 30 wt. %, in a first dewatering step, and then to about 35 wt. %, about 40 wt. %, about 45 wt. %, about 50 wt. %, about 55 wt. %, about 60 wt. %, or about 65 wt. %, and ranges thereof, such as about 35 to about 65 wt. %, or from about 35 to about 60 wt. %, or from about 40 to about 55 wt. %, in a second dewatering step. The liquid stream from the acid-impregnated cellulosic biomass dewatering step may suitably be recycled to the pulper, used as a wash stream for the heavy and coarse contaminants, used as a wash stream for the second oversize fraction and/or be recycled to acid impregnation.

In some optional aspects, supplemental cellulosic waste, such as solid food waste, lawn and garden waste, tree pruning, agricultural residues (straw, corn stover, husks), grasses, and/or wood may be combined with the cellulosic waste and acid in either solid phase mixing or acid impregnation slurry aspects. Suitable secondary sources of cellulose waste include shredded paper and yard waste such as leaves, and grass clippings. Generally, if supplemental cellulose waste is present, the ratio thereof to solid waste is not narrowly critical and is generally selected to achieve a total cellulose content of at least about 25 wt. %, at least about 30 wt. %, at least about 35 wt. %, at least about 40 wt. %, at least about 45 wt. %, or at least about 50 wt. % in the solids fraction of treated cellulosic waste as described in more detail below. In such aspects, as depicted in FIGS. 1 and 2, the secondary waste source 54 is commuted in suitable particles size reduction equipment 55 to generate milled or ground secondary cellulosic biomass 56 wherein a plurality of the particles contained therein are characterized as having a particle size distribution such that no more than about 40 wt. %, no more than about 30 wt. %, or no more than about 20 wt. % of the particles are retained by a #10 sieve. Additionally or alternatively, commuted secondary cellulosic biomass suitable for use in the process of the present invention may comprise particles of a size distribution such that at least about 60 wt. %, at least about 70 wt. %, or at least about 80 wt. % of the particles are retained by a #60 sieve. In any of the various commuting aspects, suitable size reduction equipment include, for instance, grinders and/or hammer mills.

### Steam Treatment

In some aspects of the present invention, treated cellulosic biomass may be subjected to elevated pressure and temperature conditions to break down the cellulose-hemicellulose and cellulose-hemicellulose-lignin complexes. Generally, as depicted in FIGS. 1 and 2, acid-impregnated biomass 57 is subjected to elevated pressure and temperature in the presence of steam 61 in a suitable reactor, or vessel 60. After a period of contact time, the pressure of the treated cellulosic biomass is reduced and/or the treated feedstock is discharged to an environment of reduced pressure, such as atmospheric pressure to generate steam treated cellulosic biomass 63 and flash and vent steam 62. The change in pressure assists in breaking down the biomass fiber structure including, for example, the bonds between lignin (if present) and hemicellulose and/or cellulose in the cellulose-hemicellulose or cellulose-hemicellulose-lignin complex (collectively termed “cellulose complexes”). More particularly, steam treatment typically dissociates cellulose from hemicellulose and lignin (if present) thereby providing cellulose suitable for enzymatic hydrolysis to glucose. For example, in various aspects, at least about 60 wt. %, at least about 70 wt. %, at least about 80 wt. %, or up to 90 wt. % of the cellulose contained in the cellulose complex is dissociated therefrom. Steam treatment also typically dissociates hemicellulose from the complex, generally in the form of hemicellulose solubilized within a liquid phase of the treated cellulosic biomass. For example, in various aspects, at least about 60 wt. %, at least about 70 wt. %, at least about 80 wt. %, or up to 90 wt. % of the hemicellulose contained in the cellulosic biomass is solubilized within a liquid phase of the treated cellulosic biomass. In this manner, steam treatment provides hemicellulose suitable for enzymatic hydrolysis to monosaccharides.

In some aspects of the present invention, acid-impregnated cellulosic biomass is generally introduced into a vessel comprising a contact zone for steam treatment. The acid-impregnated cellulosic biomass is typically in the form of a slurry, or cake. For example, an acid-impregnated slurry may be pressed to form a cake, or plug of treated solids for introduction into the steam treatment vessel. The precise form and configuration of the vessel is not narrowly critical and may be selected by one skilled in the art depending on the particular circumstances (e.g., properties of the cellulosic biomass and operating conditions). Generally, the vessel includes an inlet for introduction of the cellulosic biomass and one or more outlets for releasing treated cellulosic biomass and/or various components generated during the steam treatment. Once the cellulosic biomass is contained in the vessel, the vessel is pressurized and the cellulosic biomass heated by direct steam injection. In some aspects of the present invention, cellulosic biomass heating can additionally be done indirectly, such as by applying steam to a vessel jacket. Typically, the cellulosic biomass is maintained at a target temperature and pressure, such as by pressure control, for a time sufficient to provide suitable heating of the cellulosic biomass. In some aspects of the present invention, after a period of pressurizing the vessel and heating the cellulosic biomass, the cellulosic biomass is released or transferred from the contact vessel to a receiving vessel having reduced and controlled pressure. In some other aspects of the present invention, after a period of pressurizing the vessel and heating the cellulosic biomass, the pressure and temperature in the vessel is reduced to an intermediate pressure and temperature and held for a period of time at those conditions, followed by pressure reduction to atmospheric pressure or a pressure slightly above atmospheric pressure. In yet some other aspects of the present invention, after a period of pressurizing the vessel and heating the cellulosic biomass, the pressure and temperature in the vessel is reduced to atmospheric pressure or a pressure slightly above atmospheric pressure. In any of the various aspects of the present invention, as noted, the abrupt decrease in pressure during this release promotes break down of the cellulose complex. That is, the abrupt decrease in pressure causes a rapid increase in volume of the steam and gases trapped inside the biomass pore structure resulting in high rapid incident gas velocities and/or rapid vaporization of heated water that has either occupied or been forced into the fiber structure. In cases where the pressure differential is sufficiently high and wherein the pressure change occurs rapidly, the concomitant rapid vaporization and gas velocity occur essentially instantaneously in a process known in the art as steam explosion. In any of the various aspects of the present invention, the depressurization step generates flash steam comprising various volatile organic compounds (“VOC”) generated as byproducts of cellulose, hemicellulose and lingo-cellulose acid and steam treatment. Examples of such VOCs include hydroxymethylfurfural (HMF), furfural and acetic acid.

In some aspects of the present invention, distribution of the moisture (e.g., water vapor of steam treatment) throughout the acid-impregnated cellulosic biomass is generally uniform or substantially uniform. Uniform moisture distribution is currently believed to promote relatively uniform temperature throughout the contact zone and relatively uniform temperature of the cellulosic biomass. Thus, typically the cellulosic biomass is brought to a target temperature within the contact zone by distribution of steam throughout the cellulosic biomass such that the average temperature of a significant portion of the cellulosic biomass does not vary from a target temperature to any significant degree. For example, in various preferred aspects of the present invention, the average temperature of a region of the cellulosic biomass (e.g., a portion of the cellulosic biomass constituting at least about 5% by weight, at least about 25% by weight, or at least about 50% by weight of the feedstock) does not differ by more than about 5° C. from the target temperature. By way of further example, the average temperature of a region of the cellulosic biomass constituting at least about 60% by weight, or at least about 75% by weight of the cellulosic biomass does not differ by more than about 5° C. or no more than about 3° C. from the target temperature. To promote even temperature distribution throughout the vessel and/or contact zone, various controls are utilized. For example, is some aspects of the present invention, the total solids content of the feedstock introduced into the vessel and/or contact zone is maintained at from about 30 wt. % to about 70 wt. % (e.g., from about 40 wt. % to about 60 wt. %). Having a cellulosic biomass of total solids content within this range promotes even heating of the acid impregnated cellulosic biomass by direct steam injection as higher moisture content cellulosic biomasses can result in formation of a large amount of condensate on the cellulosic biomass that may hinder steam penetration and heat transfer throughout the cellulosic biomass. In addition, multiple steam nozzles may be utilized to promote relatively quick injection of steam into the treatment vessel. For example, in connection with batch treatment digesters preferably multiple steam nozzles are placed at lower portions of the digester and others are placed at the height of the reactor such that when the valves are opened initially there will be direct contact between steam and feedstock mass settled in the vessel. In some aspects of the present invention, as least a portion of the steam is introduced to the contact zone subsurface to the cellulosic biomass.

Generally, the proportion of steam utilized depends on the initial moisture content, temperature, and/or void volume of the cellulosic biomass, as well as the desired treatment temperature and pressure. Typically, the ratio of the total mass of H₂O (i.e., steam) to cellulosic biomass (dry matter basis) introduced into the vessel and/or contact zone is at least about 0.3:1, more typically at least about 0.4:1 and, still more typically, at least about 0.5:1. For example, in various preferred aspects of the present invention, the mass ratio of H₂O to acid-impregnated cellulosic biomass (dry matter basis) is from about 0.2:1 to about 0.5:1, from about 0.4:1 to about 1:1, or from about 0.3:1 to about 0.7:1, resulting in condensation of super atmospheric water vapor which intermingles with and penetrates the fiber.

Generally, steam is introduced into the vessel under a pressure of at least about 518 kPa (75 psig), at least about 863 kPa, or at least about 1035 (kPa). Typically, acid-impregnated cellulosic biomass and steam are contacted within a contact zone comprising an inlet comprising a receiving zone for treated cellulosic biomass and an outlet for removal of cellulosic biomass from the contact zone under a pressure of slightly above atmospheric (e.g. about 7 to about 21 kPa), about 35 kPa, about 173 kPa, about 345 kPa, about 518 kPa, 690 kPa, 863 kPa, 1035 kPa, 1208 kPa, 1380 kPa, 1553 kPa or about 1725 kPa, and ranges thereof, such as from about 35 kPa to about 350 kPa, from about 518 kPa to about 1725 kPa, from about 621 kPa to about 1450 kPa, or from about 1035 kPa to about 1380 kPa. The contact zone temperature is about 150° C., about 160° C., about 170° C., about 180° C., about 190° C., about 200° C. or about 210° C. The contact zone treatment time may suitably be about 0.5 minute, about 1 minute, about 2 minutes, about 3 minutes, about 4 minutes, about 5 minutes, about 10 minutes, about 15 minutes, about 20 minutes, about 25 minutes or about 30 minutes, and ranges thereof, such as from about 0.5 minute to about 30 minutes, from about 0.5 minute to about 15 minutes, from about 1 minute to about 15 minutes, from about 1 minute to about 10 minutes, from about 2 minutes to about 6 minutes, or from about 3 minutes to about 5 minutes. Steam can suitably be saturated or super-saturated, while in some aspects of the present invention the steam is saturated.

After exposure to the high pressure and high temperature conditions, the treated cellulosic biomass is released from the contact zone to a depressurization zone having lower pressure, such as in a receiving vessel, or the pressure in the contact zone in reduced. Pressure changes associated with such treatment may suitably be about 138 kPa (20 psig), about 207 kPa, about 276 kPa, about 345 kPa, about 414 kPa, about 483 kPa, about 552 kPa, about 621 kPa, about 690 kPa, about 690 kPa, about 690 kPa, about 759 kPa, about 828 kPa, about 897 kPa, about 966 kPa, about 1035 kPa, about 1104 kPa, about 1173 kPa, about 1242 kPa, about 1311 kPa, about 1380 kPa, about 1449 kPa or about 1518 kPa, and ranges thereof, such as from about 207 to about 1518 kPa, from about 345 to about 1380 kPa, from about 518 to about 1380 kPa, from about 690 to about 1380 kPa, from about 690 to about 1208 kPa, from about 863 to about 1380 kPa, from about 863 to about 1208 kPa, from about 1035 to about 1380 kPa, or from about 1035 to about 1208 kPa.

In some aspects of the present invention, steam treatment is single-stage wherein the pressure is reduced in the contact zone or in a depressurization zone to essentially atmospheric in less than about 60 seconds, less than about 40 seconds, less than about 30 seconds, less than about 20 seconds, less than about 10 seconds, less than about 5 seconds, less than about 4 seconds, less than about 3 seconds or less than about 2 seconds in a steam explosion reaction.

In some other aspects of the present invention, steam treatment comprises two-stages, comprising a first treatment stage and a second treatment stage. In preferred two-stage treatment aspects of the present invention, the first treatment stage is conducted at a temperature and pressure sufficient to achieve an activation energy sufficient to break bonds between lignin, hemicellulose and/or cellulose and provide cellulose and hemicellulose for hydrolysis to fermentable sugars. The first treatment stage time is preferably limited to prevent significant cellulose and sugar degradation. In a second step, the pressure of the biomass from the first step is reduced and controlled to levels above atmospheric conditions sufficient to provide a temperature necessary to achieve substantial hemicellulose hydrolysis to fermentable sugars without excessive degradation of cellulose and sugars.

The first treatment stage is conducted at a gauge pressure of at least about 345 kPa and a temperature of at least about 150° C. and the second treatment stage is conducted at a pressure differential of at least about 35 kPa less than the first treatment stage pressure and is controlled above atmospheric pressure. Following the second treatment step, the pressure is reduced to essentially atmospheric. In some aspects of the present invention, the pressure differential between the first and second treatment stages is about 69 kPa, about 138 kPa, about 207 kPa, about 276 kPa, about 345 kPa, about 414 kPa, about 483 kPa, about 552 kPa, about 621 kPa or about 690 kPa. In some aspects, the first treatment stage gauge pressure is from about 1035 kPa to about 1725 kPa, the first treatment stage temperature is from about 185° C. to about 208° C., the second treatment stage gauge pressure is from about 35 kPa to about 345 kPa, from about 173 kPa to about 345 kPa, from about 35 kPa to about 965 kPa, from about 173 kPa to about 965 kPa, from about 345 kPa to about 965 kPa, from about 518 kPa to about 965 kPa, from about 690 kPa to about 965 kPa, from about 345 kPa to about 828 kPa, or from about 345 kPa to about 690 kPa and the second treatment stage temperature is from about 108° C. to about 148° C., from about 131° C. to about 148° C., from about 108° C. to about 183° C., from about 131° C. to about 183° C., from about 148° C. to about 183° C., from about 160° C. to about 183° C., from about 170° C. to about 183° C., from about 148° C. to about 177° C., or from about 148° C. to about 170° C. In some other aspects, the first treatment stage gauge pressure is from about 1208 kPa to about 1725 kPa, the first treatment stage temperature is from about 192° C. to about 208° C., the second treatment stage gauge pressure is from about 35 kPa to about 345 kPa, from about 173 kPa to about 345 kPa, from about 35 kPa to about 1104 kPa, from about 173 kPa to about 1104 kPa, from about 345 kPa to about 1104 kPa, from about 518 kPa to about 1104 kPa, from about 690 kPa to about 1104 kPa, from about 345 kPa to about 966 kPa, from about 345 kPa to about 828 kPa, from about 863 kPa to about 1104 kPa, or from about 345 kPa to about 690 kPa, and the second treatment stage temperature is from about 108° C. to about 148° C., from about 131° C. to about 148° C., from about 108° C. to about 188° C., from about 131° C. to about 188° C., from about 148° C. to about 188° C., from about 160° C. to about 188° C., from about 170° C. to about 188° C., from about 148° C. to about 183° C., from about 148° C. to about 177° C., from about 178° C. to about 188° C., or from about 148° C. to about 170° C. The first and second treatment stage treatment times may suitably each be about 0.5 minute, about 1 minute, about 2 minutes, about 3 minutes, about 4 minutes, about 5 minutes, about 10 minutes, about 15 minutes, about 20 minutes, about 25 minutes or about 30 minutes, and ranges thereof, such as from about 0.5 to about 30 minutes, from about 0.5 to about 15 minutes, from about 1 to about 15 minutes, from about 1 to about 10 minutes, from about 2 to about 6 minutes, or from about 3 to about 5 minutes. In some particular aspects, the first treatment stage pressure is from about 1208 kPa to about 1725 kPa, the first treatment stage temperature is from about 192° C. to about 208° C., the second treatment stage pressure is from about 690 kPa to about 1035 kPa and the second treatment stage temperature is from about 170° C. to about 186° C. In some other particular aspects the first treatment stage pressure is from about 1035 kPa to about 1725 kPa, the first treatment stage temperature is from about 170° C. to about 208° C., the second treatment stage pressure is from about 35 kPa to about 345 kPa, and the second treatment stage temperature is from about 108° C. to about 148° C.

In some aspects of the present invention, the two-stage treatment process is a continuous process. In such aspects, the first stage treatment pressure is from about 380 kPa to about 1725 kPa, from about 690 kPa to about 1553 kPa, from about 690 kPa to about 1380 kPa, from about 863 kPa to about 1380 kPa, from about 1035 kPa to about 1380 kPa, or from about 1035 kPa to about 1380 kPa; the first treatment stage temperature is from about 151° C. to about 208° C., from about 170° C. to about 203° C., from about 170° C. to about 198° C., from about 177° C. to about 198° C., from about 186° C. to about 198° C., or from about 192° C. to about 198° C.; the second stage treatment pressure is from about 35 kPa to about 345 kPa, from about 35 kPa to about 276 kPa, from about 35 kPa to about 207 kPa, from about 35 kPa to about 104 kPa, or from about 173 kPa to about 345 kPa; and the second treatment stage temperature is from about 108° C. to about 148° C., from about 108° C. to about 140° C., from about 108° C. to about 134° C., from about 108° C. to about 121° C., or from about 131° C. to about 148° C. In some particular aspects, the first stage pressure is from about 150 to about 200 psig and the second stage pressure is from about 5 to about 50 psig, or the first stage pressure is from about 175 to about 200 psig and the second stage pressure is from about 5 to about 50 psig.

In some aspects of the present invention the two-stage treatment process is a batch process. In such aspects, the first stage treatment pressure is about the first stage treatment pressure is from about 380 kPa to about 1725 kPa, from about 690 kPa to about 1553 kPa, from about 690 kPa to about 1380 kPa, from about 863 kPa to about 1380 kPa, or from about 1035 kPa to about 1380 kPa; the first treatment stage temperature is from about 151° C. to about 208° C., from about 170° C. to about 203° C., from about 170° C. to about 198° C., from about 177° C. to about 198° C., from about 186° C. to about 198° C., or from about 192° C. to about 198° C.; the second stage treatment is conducted at a pressure differential of about 35 kPa, 69 kPa, 173 kPa, 345 kPa, 518 kPa or about 690 kPa less than the first treatment stage pressure; the second stage treatment pressure is from about 345 kPa to about 1208 kPa, from about 690 kPa to about 1208 kPa, or from about 863 kPa to about 1208 kPa; and the second stage treatment temperature is from about 148° C. to about 192° C., from about 170° C. to about 192° C. or from about 178° C. to about 192° C. In some particular aspects, the first stage pressure is from about 1380 to about 1553 kPa and the second stage pressure is from about 690 to about 1208 kPa, or the first stage pressure is from about 1208 to about 1380 kPa and the second stage pressure is from about 690 to about 1035 kPa. After the second stage, the pressure is reduced to from atmospheric pressure to less than about 35 kPa, from atmospheric pressure to about 28 kPa, from atmospheric pressure to about 21 kPa, from atmospheric pressure to about 14 kPa, or from atmospheric pressure to about 7 kPa after the second treatment stage, wherein the pressure reduction comprises releasing at least a portion of the volatilized fraction.

In some aspects of the present invention, first treatment stage and second treatment stage are conducted in the same zone in one vessel. In some other aspects of the present invention, the first treatment stage and second treatment stage are conducted in separate zones in one vessel. In yet other aspects of the present invention, the first treatment stage and second treatment stage are conducted in separate vessels.

Pressure and temperature control in the steam treatment process can be achieved by regulating steam input to the contact zone, depressurization zone, first treatment stage, second treatment stage, or a combination thereof. Pressure and temperature control may also be done by venting volatilized fraction comprising steam and VOCs from the contact zone, depressurization zone, first treatment stage, second treatment stage, or a combination thereof. Pressure and temperature control may also be achieved by a combination of steam input regulation and venting. Such pressure and temperature control may be suitably accomplished in either single stage steam explosion reaction or in two-stage steam treatment regimen. In any such aspect, the volatilized fraction may be continuously released from the treatment reactor in single stage treatment process schemes or from the first treatment stage and/or second treatment stage in two stage treatment process schemes. Alternatively, the volatilized fraction may be discontinuously released from the treatment reactor in single stage systems or from the first stage and/or second treatment stage in two stage treatment systems. As used herein, discontinuous refers to period venting. Further, in two stage treatment systems, continuous or discontinuous venting may be independent done in either of the first or second treatment stages. Methods of pressurizing vessels and venting pressurized vessels, such as by a pressure control loop comprising a pressure sensor, pressure transmitter, a pressure controller, and a pressure control valve, are known to those skilled in the art.

The volatile fraction of the cellulosic biomass typically comprises the VOC degradation products of cellulose, hemicellulose and lingo-cellulose that are volatile at temperatures in excess of about 110° C. In acid treatment aspects of the present invention, the volatile fraction is acidic. The composition of the volatilized fraction varies with the composition of the solid waste feedstock, including HMF, furfural and/or acetic acid. Such VOCs may react with solubilized sugars and/or form inhibitors of enzymatic hydrolysis Venting at least a portion of the volatilized fraction allows for removal of VOCs that would otherwise react with solubilized sugars and reduce fermentable sugar content and/or form inhibitors of enzymatic hydrolysis. As depicted in FIGS. 1 and 2, in acid treatment aspects of the present invention, the flash steam and/or treatment vent steam 62, each comprising VOCs and having a pH below about 4, are routed to the heat recovery system 80 and condensed to form the acid condensate 81. The steam condensate, containing condensed VOCs, may be recycled to the pulper 10. Therein, VOCs, such as furfural and acetic acid, react with paper filler such as, for instance, calcium carbonate, clays, or other inorganic compounds or alkali contaminants, and are subsequently predominantly purged from the process, such as in the fine contaminants stream 42. Flash steam and/or treatment steam recycle provides numerous advantages. For instance, process water use requirements are reduced. Further, heat recovery allows for minimizing the energy required to raise the pulping temperature to within a desirable operating range. Yet further, reaction of VOCs with solid waste contaminants and subsequent removal from process as solid waste reduces the toxicity and biological oxygen demand (“BOD”) loading to the process wastewater system. Still further, it is believed that the VOCs dissolve a portion of the calcium carbonate or other contaminant components present in the solid waste thereby enhancing enzyme accessibility to cellulose and hemicellulose and facilitating removal of those contaminants, which may otherwise inhibit enzyme activity, from the process. Yet further, the pH of the steam condensate streams serves to lower the pH of the pulping slurry thereby maximizing acid usage.

Steam treatment of cellulosic biomass generally results in process streams comprising a solids fraction, a liquid fraction and the volatilized fraction. The solids fraction of the treated cellulosic biomass generally comprises the solids of the acid-impregnated feedstock that are not solubilized (i.e., are water-insoluble) or volatilized during acid and steam treatment. The composition of the solids fraction varies with the composition of the solid waste feedstock, but generally comprises cellulose, unsolubilized lignin, unsolubilized hemicellulose, and unsolubilized ash. Typically, the solids fraction generally constitutes at least about 30 wt. %, at least about 40 wt. %, or at least about 50 wt. % of the treated feedstock. For example, typically the water-insoluble solids fraction constitutes from about 40 wt. % to about 80 wt. % of the treated feedstock, more typically from about 50 wt. % to about 75 wt. % and, still more typically, from about 60 wt. % to about 75 wt. % of the treated cellulosic biomass. Generally, cellulose constitutes at least about 25 wt. %, at least about 30 wt. %, or at least about 40 wt. % of the solids fraction, such as from about 30 wt. % to about 50 wt. %, more typically from about 35 wt. % to about 45 wt. % and, still more typically, from about 30 wt. % to about 40 wt. % of the solids fraction. In any of the various aspects of the present invention, the solids fraction typically comprises at least about 40 wt. %, more typically at least about 45 wt. % and, still more typically, at least about 50 wt. % of the initial cellulose content of the solid waste. The solids fraction also typically comprises from about 10 wt. % to about 40 wt. %, or from about 20 wt. % to about 30 wt. % lignin. The solids fraction typically comprises up to about 75 wt. % or up to about 95 wt. % of the initial lignin content of the solid waste feedstock. The solids fraction also further typically comprises from about 25 wt. % to about 45 wt. %, or from about 30 wt. % to about 40 wt. % glucan. Treatment generally solubilizes a significant fraction of hemicellulose, but a fraction of hemicellulose may be present in the water-insoluble solids fraction. For example, hemicellulose may constitute up to about 4 wt. %, up to about 6 wt. %, or up to about 8 wt. % of the water-insoluble solids fraction. More particularly, up to about 6 wt. %, up to about 10 wt. %, up to about 20 wt. %, or up to about 25 wt. % of the initial hemicellulose content of the solid waste feedstock may be present in the solids fraction. The solids fraction also typically comprises less than about 15 wt. %, less than about 10 wt. % or less than about 8 wt. % ash The solids fraction typically further comprises various other polysaccharides including, for example, starch, dextrose, xylan, arabinan, mannan, galactan, and combinations thereof, and various monosaccharides such as glucose, fructose, xylose, arabinose, mannose, and combinations thereof.

The liquid fraction of the treated cellulosic biomass typically comprises solubilized hemicellulose, solubilized cellulose, solubilized components provided by degradation of lignin, and fermentable sugars. The composition of the liquid fraction varies with the composition of the solid waste feedstock, but the fermentable sugars (e.g., glucose, xylose, arabinose, mannose, galactose, and various oligomers thereof) generally constitute at least about 30 wt. %, at least about 50 wt. %, or at least about 75 wt. % of the content of the liquid fraction on a total solids basis. Typically, fermentable sugars typically constitute from about 30 to about 95 wt. %, from about 30 to about 75 wt. % or from about 50 to about 75 wt. % of the of the content of the liquid fraction on a total solids basis. Lignin typically constitutes at least about 0.5 wt. %, at least about 1 wt. % and, or at least about 4 wt. % of the liquid fraction on a total solids basis. Additionally or alternatively, the liquid fraction may also comprise water-soluble lignin-derived phenolic components and relatively low molecular weight lignin degradation products.

### Conditioning and Lignin Recovery

In some optional aspects of the present invention, the treated cellulosic biomass may be conditioned to reduce the concentration of one or more components that may inhibit enzymatic hydrolysis of cellulose and/or hemicellulose. For example, lignin is often broken down into water-soluble phenolic enzymatic inhibitor compounds during acid and/or steam treatment; said inhibitors can be inactivated or extracted from the treated cellulosic biomass. In some other optional aspects of the present invention, lignin, an enzyme inhibitor, may be recovered from the treated cellulosic biomass solids fraction prior to the enzymatic conversion thereof to monosaccharides. More particularly, lignin (such as generated in the break-down of the cellulose-hemicellulose-lignin complex) may be separated from cellulose, hemicellulose, polysaccharides (e.g., glucan and starch) and monosaccharides, and recovered from the treated cellulosic biomass. Treated cellulosic biomass may also comprise degradation products of hemicellulose and/or cellulose hydrolysis. For example, during treatment hemicellulose and/or cellulose may be hydrolyzed to form a sugar that may be degraded to form one or more of HMF, furfural, and/or acetic acid. It is currently believed that conditioning and/or removal of enzyme inhibitors contributes to improved fermentable sugar and yield of fermentation products from hemicellulose and cellulose. In such aspects of the present invention, treated feedstock is typically conditioned in the absence of any intermediate steps between steam treatment and addition of conditioning agents. However, since further degradation products may form in the treated feedstock at elevated temperature, the temperature of the feedstock prior to conditioning is preferably maintained at no more than about 140° C., or no more than about 120° C. If necessary, the treated feedstock may be cooled prior to conditioning to the preferred temperature range.

In any of the various conditioning and lignin recovery aspects the biomass for conditioning may suitably be in the form of a slurry or a solids fraction having a total solids content of at least about 10 wt. %, at least about 20 wt. %, or at least about 30 wt. %. For example, typically the solids content of the biomass for conditioning is from about 10 wt. % to about 50 wt. % and, still more typically, from about 20 wt. % to about 40 wt. %.

In some lignin recovery aspects of the present invention, the treated cellulosic biomass solids are introduced into a lignin extraction vessel or device and contacted with an extraction solvent in which lignin is soluble to form extraction mixture comprising a liquid fraction comprising lignin (e.g., lignin dissolved in the extraction solvent) and a solid phase fraction comprising cellulose and other polysaccharides and monosaccharides that is depleted in lignin relative to the solids fraction. In some aspects of the present invention, enzymatic inhibitors such as lignin may be extracted from the treated cellulosic biomass at high pH with an alkali metal hydroxide such as sodium hydroxide, potassium hydroxide, ammonium hydroxide, calcium oxide (lime) or a combination thereof. Advantageously, alkali metal hydroxides further form phenate salts from the phenolic inhibitors. Such salts have reduced inhibitor effect and may be extracted from the treated cellulosic biomass. In various preferred alkali metal hydroxide aspects of the invention, the extraction solvent comprises sodium hydroxide dissolved in water and, more particularly, is in the form of an aqueous solution of sodium hydroxide containing sodium hydroxide at a concentration of from about 0.5 wt. % to about 2 wt. %, or from about 0.5 wt. % to about 1 wt. %, and a pH of from about 10 to about 14, such as about 13. In some aspects, the extraction solvent is an organic solvent comprising, for instance, methanol, ethanol, butanol, acetone, and combinations thereof. In some preferred organic solvent aspects of the present invention, the extraction solvent is ethanol. In any of the various aspects, the extraction temperature is suitably from about 30° C. to about 60° C., or from about 40° C. to about 50° C. However, neither the conditions of nor the manner of contact of the solids fraction with the extraction solvent are narrowly critical and are generally conducted in accordance with conventional methods known in the art. See, for example, Canadian Patent Nos. 1,267,407 and 1,322,366, and U.S. Pat. Nos. 3,817,826, 4,470,851, 4,764,596, 4,908,099, and 4,966,650, the entire contents of which are incorporated herein by reference for all relevant purposes. The extraction mixture is then processed using any suitable solid-liquid separation device known in the art, as described elsewhere herein, including, for example, a screen, filter, centrifuge, settler, screw press or belt press to generate a liquid lignin fraction and a solids fraction. The lignin fraction comprises at least about 1 wt. %, about 2 wt. % or at least about 3 wt. % lignin, such as from about 1 wt. % to about 10 wt. %, or from about 2 wt. % to about 6 wt. % lignin. Generally at least about 40 wt. %, about 50 wt. %, about 60 wt. %, or at least about 70 wt. % of the lignin present in the treated cellulosic biomass is extracted into the lignin fraction.

Lignin may optionally be recovered from the lignin fraction. Lignin recovery generally proceeds in accordance with conventional methods known in the art (e.g., precipitation) as described, for example, in U.S. Pat. No. 4,966,650, the entire contents of which are incorporated herein by reference for all relevant purposes. In some of the various aspects, acid and the lignin fraction are introduced into a suitable vessel for precipitation of the lignin-rich solids from the lignin extract. Relatively concentrated acid is generally preferred. For example, the acid may be in the form of sulfuric acid having a concentration of at least about 50 wt. %, at least about 80 wt. %, or at least about 90 wt. % sulfuric acid. The precipitated lignin separated from the acidic lignin mother liquor by any suitable solid-liquid separation method as described elsewhere herein, such as by filtration. In some aspects, the recovered lignin is dried to for powdered lignin composition. Lignin-rich compositions are suitable for use in a variety of applications including, for example, as a phenol formaldehyde resin extender in the manufacture of particle board and plywood, manufacture of molding compounds, urethane and epoxy resins, antioxidants, feeds, fuels, pelletizing aids, drilling mud stabilizers, and cement additives. The acidic lignin mother liquid may suitably be recycled to the pulper and/or to the acid impregnation system.

In some aspects, the treated cellulosic biomass and/or its related liquid fraction may be contacted with protein-containing material that will absorb the phenolic compounds and/or form a complex and/or adduct with the phenolic compounds. Various protein-containing materials (e.g., enzymes, yeast cells and fermentation broths generated during enzyme production) are suitable for this purpose. Enzymes (e.g., lacase) may provide degradation of phenolic compounds. In addition, protein-containing material derived at other process stages may be utilized. For example, thin stillage produced as described elsewhere herein may be used for this purpose. Metal salts and/or protein-containing materials may also be used in treatment for the purpose of complexing and/or absorbing hemicellulose and/or cellulose degradation products. Suitable metal salts (e.g., ferrous sulfate and magnesium sulfate) may be introduced into the liquor fraction of the treated feedstock at a concentration of from about 0.05 to about 1 millimole/L.

In other aspects the treated cellulosic biomass may be adjusted to a pH range suitable for enzymatic hydrolysis, such as from about 4 to about 6.5, from about 4.5 to about 6, or from about 5 to about 5.5. The adjusted treated cellulosic biomass is then extracted with process water and/or a suitable recycle stream such as the recycled water stream 41 to extract enzyme inhibitors. The wash stream may be suitably recycled to the pulper 10.

Conditioning agents are typically in the form of an aqueous liquid medium comprising one or more of the above noted conditioning agents. Typically, one or more of such agents are present in the stream at a proportion of from about 0.25 to about 2.5 wt. % and, more typically, at a proportion of from about 0.5 to about 1 wt. %. Generally, the conditioning agent medium to treated cellulosic biomass introduced into conditioning equipment is at least about 0.05:1, or at least about 0.1:1. For example, typically the mass ratio of the conditioning agent medium to treated cellulosic biomass introduced into the conditioning vessel is from about 0.05:1 to about 0.25:1 and, more typically from about 0.1:1 to about 0.2:1. Contact of the treated cellulosic biomass with the conditioning stream within the conditioning equipment forms conditioned cellulosic biomass. With respect to the principal components of value, i.e., cellulose, hemicellulose, and sugars, the composition of the conditioned cellulosic biomass generally corresponds to that of the treated cellulosic biomass, with the proportions of the components reduced based on dilution of the treated cellulosic biomass by mixing with the conditioning stream within the conditioning vessel. It is currently believed that conditioning has little, if any, impact on, for example, the cellulose, hemicellulose, solubilized sugar and/or lignin composition of the cellulosic biomass.

### Enzyme Hydrolysis

The treated cellulosic biomass, conditioned treated cellulosic biomass, or lignin depleted cellulosic biomass, or a combination thereof, is combined with a source of enzymes comprising at least one cellulase to generate a composition comprising monosaccharides. In some aspects of the present invention, the source of enzymes further comprises at least one hemicellulase, an α-amylase, a β-amylase, a protease, and combinations thereof. In reference to FIGS. 1 and 2, the treated cellulosic biomass 63 is contacted in an enzymatic hydrolysis vessel 70 with an enzyme source 71 comprising at least one cellulase to form a hydrolyzed cellulosic composition comprising glucose 72.

Cellulases are a class of enzymes produced chiefly by fungi, bacteria, and protozoans that catalyze the cellulolysis (hydrolysis) of cellulose into glucose, cellobiose, cellotriose, cellotetrose, cellopentose, cellohexose, and longer chain cellodextrins. Combinations of the three basic types of cellulases may be employed. For example, endo-cellulases may be added to randomly hydrolyze internal β-1,4,-D-glucosidic linkages in order to disrupt the crystalline structure of cellulose and expose individual cellulose chains. Exo-cellulases may be added to cleave off two units (cellobiose), three units (cellotriose), or four units (cellotetrose) from the exposed chains, while β-glucosidase may be added to hydrolyze these compounds into glucose, which is available for fermentation. Examples of cellulases suitable for use in the present invention include, for example, Cellic® CTEC2, Cellic® CTEC3, CELLUCLAST®, CELLUZYME®, CEREFLO® and ULTRAFLO® (available from Novozymes A/S), LAMINEX®, SPEZYME®CP (Genencor Int.), and ROHAMENT® 7069 W (Rohm GmbH), and GC-220 (Genencor International).

In general, a slurry is formed from the treated cellulosic biomass at conditions favorable for cellulase activity. More particularly, the pH is preferably adjusted to from about 4 to about 6.5, from about 4.5 to about 6, or from about 5 to about 5.5. Examples of bases for pH adjustment include sodium hydroxide and ammonia and examples of suitable acids for pH adjustment include mineral acids such as sulfuric acid, nitric acid and hydrochloric acid. In some aspects of the present invention, the temperature of the adjusted liquefied mash is adjusted from about 35° C. to about 70° C., from about 45° C. to about 65° C., or from about 50° C. to about 60° C. The solids content is preferably adjusted to about 10 wt. %, about 15 wt. %, about 20 wt. %, about 25 wt. % or about 30 wt. % TS, and ranges thereof, such as from about 15% to about 25% TS or from about 18% to about 22% TS, with one or more of process water, or a recycle stream such as from water treatment. The cellulase enzymes are added in amounts effective from about 0.001% to about 5.0% wt. of solids, more preferably from about 0.025% to about 4.0% wt. of solids, and most preferably from about 0.005% to about 2.0% wt. of solids. Cellulase slurry loading may suitably vary with treated cellulosic biomass cellulose content, but typical loading may be expressed as about 5 mg, about 10 mg, about 15 mg, about 20 mg, about 25 mg, about 30 mg, about 35 mg, about 40 mg, about 45 mg or about 50 mg, and ranges thereof, such as from about 5 mg to about 50 mg, from about 10 mg to about 50 mg, from about 20 mg to about 50 mg, from about 10 mg to about 50 mg, from about 10 mg to about 40 mg, from about 10 mg to about 30 mg, from about 20 mg to about 50 mg or from about 20 mg to about 40 mg cellulase per gram of cellulose. Alternatively expressed, cellulase loading is about 5 mg, about 10 mg, about 20 mg, about 30 mg, or about 40 mg, and ranges thereof, such as from about 10 to about 40 mg enzyme protein per gram of cellulose in treated cellulosic biomass.

Cellulase may be combined with the treated cellulosic biomass by any means known in the art to achieve a substantially homogeneous admixture, including agitated mixing tanks, in line mixers, pug mill mixers, paddle mixers, ribbon mixers, or in liquefaction reactors such as reactors having at least one mixing section and at least one plug flow section. The enzymatic hydrolysis reactor is typically an agitated and vessel designed to hold the treated cellulosic biomass-cellulase mixture at a temperature suitable for cellulose hydrolysis by cellulase, wherein the volume is sufficient to provide a hold time required for a significant yield of cellulose-derived hexose monosaccharide (“C6”) sugars, e.g., glucose. In some aspects of the present invention, the enzymatic hydrolysis vessel may be insulated and/or heated with a heating jacket to maintain hydrolysis temperature. Total enzymatic hydrolysis cycle times of 48 hours, 54 hours, 60 hours, 66 hours, 72 hours, 78 hours, 96 hours and 144 hours, and ranges thereof, are within the scope of the present invention. Glucose yields, based on total cellulose content of the treated cellulosic biomass, of at least about 30%, at least about 40%, at least about 50%, at least about 60%, at least about 70% or at least about 80%, and ranges thereof, such as from about 30% to about 90%, from about 40% to about 80% from about 30% to about 70% or from about 60% to about 75% of theoretical value are achieved. Alternatively expressed, the total yield of glucose in the hydrolyzed cellulose composition is about 0.03, about 0.05, about 0.07, about 0.09 or about 0.13 grams, and ranges thereof, of glucose per mg cellulase.

For highly viscous treated cellulosic biomass slurry, such as having a viscosity in excess of about 20,000 cP, about 30,000 cP, about 50,000 cP, about 60,000 cP or even about 100,000 cP, mixing can be done in two stages. In a first stage, cellulase can be admixed with the treated cellulosic biomass in a mixer particularly suited for the processing of highly viscous materials, for instance, a pug mill mixer, a paddle mixer (single or double shaft), or a ribbon mixer (single or double shaft). High viscosity mixers are particularly suited to the process of the present invention because thorough mixing of cellulase with the viscous treated cellulosic biomass slurry enables a rapid viscosity reduction in the subsequent liquefaction step where the viscosity is preferably reduced to less than about 20,000 cP, less than about 15,000 cP, less than about 10,000 cP or even less than about 5,000 cP. The high viscosity mixer may optionally have a jacket to receive cooling or heating medium in order to maintain the temperature of the treated cellulosic biomass during cellulase addition. Optionally, cooling and heating medium may be incorporated into the internal mixer components (such as rotating shafts, paddles) to further enhance heat exchange. In some aspects, cellulase addition can be done through one or more addition points, for example, multiple spray nozzles, position near the treated cellulosic biomass inlet. In a second stage, the treated cellulosic biomass-cellulase admixture may be processed in a mix tank or fiber liquefaction bioreactor. In some aspects, the treated cellulosic biomass-cellulase admixture may be processed in a fiber liquefaction bioreactor to further reduce the viscosity prior to transfer to a cellulose hydrolysis reactor. The fiber liquefaction bioreactor may be of either a continuous mixing design or a design having at least one continuous mixing section and at least one plug flow section. Optionally, two or more fiber liquefaction bioreactors may be operated in series. In some particular aspects, the fiber liquefaction bioreactor comprises alternating mixing zones and near plug flow zones and the treated cellulosic biomass-cellulase admixture either flows downward through the tower by gravity or is moved upward by pumping. The treated cellulosic biomass-cellulase admixture is typically process in a fiber liquefaction bioreactor until the admixture viscosity is less than about 10,000 cP, less than about 9,000 cP, less than about 8,000 cP, less than about 7,000 cP or less than about 5,000 cP where after it is transferred to a cellulose hydrolysis reactor.

Optionally, additional enzymes such as a hemicellulase (e.g., a xylanase), an α-amylase, a β-amylase, a glucoamylase, an arabinoxylanase, a pullulanase, and/or a protease can be added to the treated cellulosic biomass to generate additional C6 sugars and/or pentose (“C5”) sugars. Non-limiting examples of C6 sugars include glucose, galactose, mannose, and fructose and non-limiting examples of C5 sugars include xylose, arabinose and ribose. The optional enzymes may be admixed with the treated cellulosic biomass at any point of hydrolysis including with the cellulase during high viscosity admixing, at one or more locations in the fiber liquefaction bioreactor and/or in the cellulose hydrolysis reactor.

Hemicellulases may be added to further hydrolyze the various types of hemicelloses. A hemicellulase, as used herein, refers to a polypeptide that can catalyze hydrolysis of hemicellulose into small polysaccharides such as oligosaccharides, or monomeric saccharides including xylose and arabinose. Hemicellulases can be placed into three general categories: the endo-acting enzymes (e.g., endo-1,4-β-D-xylanases) that hydrolyze internal bonds within the polysaccharide (xylan) chain; the exo-acting enzymes (e.g., 1,4-β-D-xylosidases) that act processively from either the nonreducing end of polysaccharide chain and liberate D-xylose residues; and accessory enzymes. Hemicellulases include, for example, the following: endoxylanases, β-xylosidases, β-L-arabinofuranosidases, β-D-glucuronidases, feruloyl esterases, coumarolyl esterases, α-galactosidases, β-galactosidases, β-mannanases, and β-mannosidases. Of the accessory enzymes, an α-L-arabinofuranosidase catalyzes the hydrolysis of terminal non-reducing α-L-arabinofuranoside residues in α-L-arabinosides. An α-glucuronidase catalyzes the hydrolysis of an α-D-glucuronoside to D-glucuronate and an alcohol. An acetylxylanesterase catalyzes the hydrolysis of acetyl groups from polymeric xylan, acetylated xylose, acetylated glucose, alpha-naphthyl acetate, and p-nitrophenyl acetate. An α-galactosidase catalyzes the hydrolysis of terminal, non-reducing α-D-galactose residues in α-D-galactosides, including galactose oligosaccharides, galactomannans, galactans and arabinogalactans. A β-galactosidase catalyzes the hydrolysis of terminal non-reducing β-D-galactose residues in β-D-galactosides. Such a polypeptide may also be capable of hydrolyzing α-L-arabinosides. A 6-mannanase catalyzes the random hydrolysis of 1,4-β-D-mannosidic linkages in mannans, galactomannans and glucomannans. A β-mannosidase catalyzes the hydrolysis of terminal, non-reducing β D mannose residues in β-D-mannosides. In some aspects, the hemicellulase is an exo-acting hemicellulase, such as an exo-acting hemicellulase which has the ability to hydrolyze hemicellulose under acidic conditions of below pH 7. A xylanase may be obtained from any suitable source, including fungal and bacterial organisms, such as Aspergillus, Disporotrichum, Penicillium, Neurospora, Fusarium, Trichoderma, Humicola, Thermomyces, and Bacillus. Commercially available preparations comprising xylanase include SHEARZYME®, BIOFEED WHEAT®, BIO-FEED Plus®L, ULTRAFLO®, VISCOZYME®, PENTOPAN MONO®BG, and PULPZYME®HC (Novozymes A/S), and LAMINEX® and SPEZYME®CP (Genencor Int.) An example of a hemicellulase suitable for use in the present invention includes VISCOZYME® (available from Novozymes A/S, Denmark). Hemicellulase loadings vary with the treated cellulosic biomass hemicellulose content and is generally about 5 mg, about 10 mg, about 20 mg or about 30 mg enzyme per gram of hemicellulose, and ranges thereof. The C5 sugar content of the treated cellulosic biomass hydrolyzate represents a yield of at least about 20%, at least about 30%, at least about 35%, at least about 40%, at least about 45% or at least about 50% based on the hemicellulose content of the treated cellulosic biomass.

An α-amylase may be added to liquefy free starch that was formerly entrapped within the cellulose, hemicellulose and/or lignocellulosic matrices. The α-amylase loading varies with the starch content in the hydrolyzed treated cellulosic biomass and is typically about 0.1 wt. %, about 0.2 wt. %, about 0.3 wt. %, or about 0.4 wt. % of the starch content, and ranges thereof, such as from about 0.1 to about 0.4 wt. % of the starch content in treated cellulosic biomass solids. A glucoamylase may be added to convert liquefied starch to C6 sugars. A pullulanase may be added to catalyze the hydrolysis of amylopectin at the 1→6 bond, thereby yielding oligomers of D-glucose. A commercially available pullulanase is Promozyme® D2, available from Novozyme Corporation.

Also useful are multienzyme complexes containing multiple carbohydrases, such as Viscozyme® L, available from Novozyme Corporation, which contains arabanase, cellulase, β-glycanase, hemicellulase, and xylanase.

Proteases may be added to hydrolyze peptide bonds that link amino acids together in polypeptide chains to form short chain polypeptides. In general, fine starch granules, may be encased in a protein matrix. Proteases are useful for hydrolyzing the peptide bonds and releasing these starch granules. Moreover, proteases enhance the solubility of proteins, oligopeptides, and amino acids. Further, short chain polypeptides can be used by yeast for biological activities. Without being bound by a particular theory, it is thought that hydrolysis of the proteins into peptides and amino acids enhances the nutritional value of material prepared from still bottoms after fermentation product (e.g., ethanol) recovery. Generally any of the classes of proteases are applicable, e.g., acid, base, or neutral, and proteases are commercially available from, for example, Novozymes, Genencor and Solvay. Examples include, for instance, GC106 (available Genencor International), AFP 2000 (available from Solvay Enzymes, Inc.), FermGen® (which is an alkaline protease available from Genencor International), and Alcalase® (which is an acid protease available from Novozymes Corporation). The amount of all acid protease is typically in the range of from about 0.01 to about 10 SAPU per gram of starch, from about 0.05 to about 5 SAPU per gram of starch, or even from about 0.1 to about 1 SAPU per gram of starch. As used herein, “SAPU” refers a spectrophotometric acid protease unit, wherein 1 SAPU is the amount of protease enzyme activity that liberates one micromole of tyrosine per minute from a casein substrate under conditions of the assay. It is believed that protease addition may increase the energy crop fermentation rate by about 5% to about 10%.

### Sugar Recovery

In some optional aspects of the present invention, monosaccharides may be extracted or separated from the hydrolyzed cellulosic biomass composition. In such aspects, hydrolyzed cellulosic biomass is introduced into a sugar recovery vessel or device which comprises suitable solids/liquid separation equipment such as, for instance, a screen, filter, centrifuge, settler, percolator, extraction column, flotation vessel, or combination thereof, to generate a liquid fraction comprising monosaccharide sugars and a solids fraction, wherein the solids fraction may suitably be in the form of a cake or concentrated slurry. In various preferred sugar recovery aspects of the present invention, the solids fraction may be washed one or more times for recovery of additional monosaccharide. In some aspects, monosaccharides may be recovered from the solid fraction by counter-current contact of the solid fraction with a washing liquid in a suitable apparatus to form a wash stream comprising extracted monosaccharides. The liquid fraction is combined with a liquid medium and/or the wash streams to form a monosaccharide fraction. The precise composition of the liquid medium and washing liquid are not narrowly critical. However, in various preferred aspects of the present invention, the liquid medium and washing liquid may be process water if a monosaccharide fraction of relatively high purity is desired or may comprise one or more process recycle streams, such as thin stillage or the recycle stream from the water treatment operation, if monosaccharide fractions of lower purity are acceptable. Although the precise composition of the monosaccharide fraction varies with the solid waste composition, generally the monosaccharide compositions comprises at least about 5 wt. %, at least about 6 wt. %, at least about 7 wt. %, at least about 8 wt. %, at least about 9 wt. %, or at least about 10 wt. % monosaccharide. The residual solids fraction comprises non-hydrolyzed cellulose, non-hydrolyzed hemicellulose, non-hydrolyzed lingo-cellulose, polysaccharides, monosaccharides and lignin. The residual solids fraction may be suitably recycled to the pulper and/or acid treatment process for the recovery of the sugars and the sugar substrates.

In some optional aspects of the present invention, the monosaccharide composition may be concentrated to produce monosaccharide concentrates or syrups having a monosaccharide content of at least about 10 wt. %, at least about 15 wt. %, at least about 20 wt. %, at least about 25 wt. %, at least about 30 wt. %, at least about 35 wt. % or at least about 40 wt. %. Concentration methods are known in the art and include evaporators and reverse osmosis.

### Fermentation

The enzyme treated biomass comprising monosaccharides can be used by suitable microorganisms as a substrate for the production of fermentation products. A wide variety of fermentation microorganisms are known in the art, and others may be discovered, produced through mutation, or engineered through recombinant means. Fermentation microorganisms within the scope of the present invention include yeast, bacteria, filamentous fungi, microalgae, and combinations thereof. In accordance with the present invention, any microorganism that utilizes fermentable sugars produced by the present process may be used to produce fermentation products. Examples of fermentation products within the scope of the present invention include, for instance, acids, alcohols, alkanes, alkenes, aromatics, aldehydes, ketones, triglycerides, fatty acids, biopolymers, proteins, peptides, amino acids, vitamins, antibiotics, pharmaceuticals, and combinations thereof. Non-limiting examples of alcohols include methanol, ethanol, propanol, isopropanol, butanol, ethylene glycol, propanediol, butanediol, glycerol, erythritol, xylitol, sorbitol, and combinations thereof. Non-limiting examples of acids include acetic acid, lactic acid, propionic acid, 3-hydroxypropionic, butyric acid, gluconic acid, itaconic acid, citric acid, succinic acid, levulinic acid, and combinations thereof. Non-limiting examples of amino acids include glutamic acid, aspartic acid, methionine, lysine, glycine, arginine, threonine, phenylalanine, tyrosine, and combinations thereof. Other examples of fermentation products include methane, ethylene, acetone and industrial enzymes.

Fermentation organisms may be wild type microorganisms or recombinant microorganisms, and include Escherichia, Zymomonas, Saccharomyces, Candida, Pichia, Streptomyces, Bacillus, Lactobacillus, and Clostridium. In some aspects of the present invention, the fermentation organism is recombinant Escherichia coli, Zymomonas mobilis, Bacillus stearothermophilus, Saccharomyces cerevisiae, Clostridia thermocellum, Thermoanaerobacterium saccharolyticum, or Pichia stipites. In some other aspects of the present invention, the microorganism is a microalgae, defined as a eukaryotic microbial organism that contains a chloroplast or plastid, and optionally that is capable of performing photosynthesis, or a prokaryotic microbial organism capable of performing photosynthesis. Microalgae include obligate photoautotrophs, which cannot metabolize a fixed carbon source as energy, as well as heterotrophs, which can live solely off of a fixed carbon source. Microalgae include unicellular organisms that separate from sister cells shortly after cell division, such as Chlamydomonas, as well as microbes such as, for example, Volvox, which is a simple multicellular photosynthetic microbe of two distinct cell types. Microalgae include cells such as Chlorella, Dunaliella, and Prototheca. Microalgae also include other microbial photosynthetic organisms that exhibit cell-cell adhesion, such as Agmenellum, Anabaena, and Pyrobotrys. Microalgae also include obligate heterotrophic microorganisms that have lost the ability to perform photosynthesis, such as certain dinoflagellate algae species and species of the genus Prototheca.

As used herein, “recombinant” is a cell, nucleic acid, protein or vector that has been modified due to the introduction of an exogenous nucleic acid or the alteration of a native nucleic acid. Thus, e.g., recombinant cells express genes that are not found within the native (non-recombinant) form of the cell or express native genes differently than those genes are expressed by a non-recombinant cell. Once a recombinant nucleic acid is made and introduced into a host cell or organism, it may replicate using the in vivo cellular machinery of the host cell.

Various fermentation microorganisms and fermentation products thereof are known in the art. Non-limiting examples include, for instance, fermentation of carbohydrates to acetone, butanol, and ethanol by: (i) solventogenic Clostridia is described by Jones and Woods (1986) Microbiol. Rev. 50:484-524; (ii) a mutant strain of Clostridium acetobutylicum is described in U.S. Pat. No. 5,192,673;and (iii) a mutant strain of Clostridium beijerinckii is described in U.S. Pat. No. 6,358,717 is known. Fermentation of carbohydrates to ethanol by modified strains of E. coli has been described by Underwood et al., (2002) Appl. Environ. Microbiol. 68:6263-6272 and by a genetically modified strain of Zymomonas mobilis is described in US 2003/0162271 A1. Preparation of lactic acid by recombinant strains of E. coli (Zhou et al., (2003) Appl. Environ. Microbiol. 69:399-407), natural strains of Bacillus (US20050250192), and Rhizopus oryzae (Tay and Yang (2002) Biotechnol. Bioeng. 80:1-12) is known. Recombinant strains of E. coli have been used as biocatalysts in fermentation to produce 1,3 propanediol (U.S. Pat. Nos. 6,013,494 and 6,514,733) and adipic acid (Niu et al., (2002) Biotechnol. Prog. 18:201-211). Acetic acid has been produced using recombinant Clostridia (Cheryan et al., (1997) Adv. Appl. Microbiol. 43:1-33) and newly identified yeast strains (Freer (2002) World J. Microbiol. Biotechnol. 18:271-275). Production of succinic acid by recombinant E. coli and other bacteria is disclosed in U.S. Pat. No. 6,159,738 and by mutant recombinant E. coli in Lin et al., (2005) Metab. Eng. 7:116-127). Pyruvic acid has been produced by mutant Torulopsis glabrata yeast (Li et al., (2001) Appl. Microbiol. Technol. 55:680-685) and by mutant E. coli (Yokota et al., (1994) Biosci. Biotech. Biochem. 58:2164-2167). Recombinant strains of E. coli have been used for production of para-hydroxycinnamic acid (US20030170834) and quinic acid (US20060003429).

Production of amino acids by fermentation has been accomplished using auxotrophic strains and amino acid analog-resistant strains of Corynebacterium, Brevibacterium, and Serratia. For example, production of histidine using a strain resistant to a histidine analog is described in Japanese Patent Publication No. 8596/81 and using a recombinant strain is described in EP 136359. Production of tryptophan using a strain resistant to a tryptophan analog is described in Japanese Patent Publication Nos. 4505/72 and 1937/76. Production of isoleucine using a strain resistant to an isoleucine analog is described in Japanese Patent Publication Nos. 38995/72, 6237/76, 32070/79. Production of phenylalanine using a strain resistant to a phenylalanine analog is described in Japanese Patent Publication No. 10035/81. Production of tyrosine using a strain requiring phenylalanine for growth, resistant to tyrosine (Agr. Chem. Soc. Japan 50 (1) R79-R87 (1976), or a recombinant strain (EP263515, EP332234), and production of arginine using a strain resistant to an L-arginine analog (Agr. Biol. Chem. (1972) 36:1675-1684, Japanese Patent Publication Nos. 37235/79 and 150381/82) have been described. Phenylalanine has also been produced by Eschericia coli strains ATCC 31882, 31883, and 31884. Production of glutamic acid in a recombinant Coryneform bacterium is described in U.S. Pat. No. 6,962,805. Production of threonine by a mutant strain of E. coli is described in Okamoto and Ikeda (2000) J. Biosci Bioeng. 89:87-79. Methionine was produced by a mutant strain of Corynebacterium lilium (Kumar et al, (2005) Bioresour. Technol. 96: 287-294). Production of peptides, enzymes, and other proteins by microorganisms is also known as disclosed in U.S. Pat. Nos. 6,861,237, 6,777,207 and 6,228,630. Production of triglycerides, fatty acids and fatty acid esters (e.g., biodiesel) by microalgae is also known as disclosed in U.S. Pat. Nos. 7,883,882, 8,187,860, 8,278,090 and 8,222,010, and published U.S. App. Nos. 20100303957, 20110047863 and 20110250658.

In one aspect of the present invention, depicted in FIGS. 1 and 2, in a fermentation vessel 90, a fermentation medium 92 is formed from comprising the enzyme treated cellulosic biomass 72, the monosaccharide fraction from extraction of the enzyme treated cellulosic biomass (not depicted in FIGS. 1 and 2), or combinations thereof, and an ethanol fermentation organism source 91 comprising at least one yeast species capable of converting glucose to ethanol. The fermentation proceeds to form a beer comprising ethanol 93. In some aspects of the present invention, the ethanol fermentation organism source further comprises one or more pentose sugar fermenting organisms and/or one or more fermenting organisms capable of converting hexose monosaccharides other than glucose to ethanol.

Selection of suitable fermentation conditions may suitably be done by those skilled in the art based on (i) the identity of the microorganism or combination of microorganisms and (ii) the associated fermentation product. Fermentation may be aerobic or anaerobic. Single and multi-step fermentations are within the scope of the present invention. The fermentation medium may be supplemented with additional nutrients required for microbial growth. Supplements may include, for example, yeast extract, vitamins, growth promoters, specific amino acids, phosphate sources, nitrogen sources, chelating agents, salts, and trace elements. Components required for production of a specific product made by a specific microorganism may also be included, such as an antibiotic to maintain a plasmid or a cofactor required in an enzyme catalyzed reaction. Also additional sugars may be included to increase the total sugar concentration. Suitable fermentation conditions are achieved by adjusting these types of factors for the growth and target fermentation product production by a microorganism. The fermentation temperature can be any temperature suitable for growth and production of the nutrients of the present invention such as from about 20° C. to about 45° C., from about 25° C. to about 40° C., or from about 28° C. to about 32° C. The fermentation pH can be adjusted or controlled by the addition of acid or base to the fermentation mixture. In such cases when ammonia is used to control pH, it also conveniently serves as a nitrogen source. The pH is maintained from about 3.0 to about 8.0, from about 3.5 to about 7.0 or from about 4.0 to about 6.5. The fermentation mixture can optionally be maintained to have a dissolved oxygen content during the course of fermentation to maintain cell growth and to maintain cell metabolism for production of the nutrients. The oxygen concentration of the fermentation medium can be monitored using known methods, such as through the use of an oxygen electrode. Oxygen can be added to the fermentation medium using methods known in the art, for, through agitation and aeration of the medium by stirring, shaking or sparging. Preferably, the oxygen concentration in an aerobic fermentation medium can be in the range of from about 20% to about 100% of the saturation value of oxygen in the medium based upon the solubility of oxygen in the fermentation medium at atmospheric pressure and at a temperature in the range of from about 20° C. to about 40° C. Periodic drops in the oxygen concentration below this range may occur during fermentation, however, without adversely affecting the fermentation. Fermentation may occur subsequent to enzymatic hydrolysis, or may occur concurrently with enzymatic hydrolysis by simultaneous hydrolysis and fermentation. In some aspects of the present invention, simultaneous hydrolysis and fermentation can keep the sugar levels produced by hydrolysis low, thereby reducing potential product inhibition of the hydrolysis enzymes, reducing sugar availability for contaminating microorganisms, and improving the conversion of treated biomass to monosaccharides and/or oligosaccharides

In aspects of the present invention directed to the generation of ethanol by yeast, the fermentation medium has a pH of from about 3.5 to about 6, from about 3.5 to about 5 or from about 4 to about 4.5 is preferred. If pH adjustment is required, mineral acids such as sulfuric acid, hydrochloric acid or nitric acid may be used, or bases such as ammonia (ammonium hydroxide) or sodium hydroxide may be used. To enhance the efficacy of ethanol fermentation and increase the ethanol yield, additional nutrients may be added to enhance yeast proliferation. Such nutrient includes, without limitation, free-amino-nitrogen (FAN), oxygen, phosphate, sulfate, magnesium, zinc, calcium, and vitamins such as inositol, pantothenic acid, and biotin. Typical sources of FAN include urea, ammonium sulfate, ammonia, amino acids, and α-amino nitrogen groups of peptides and proteins. Added FAN content is preferably from about 1.2 to about 6 mg N/g starch, for example 1.2, 2.4, 3.6, 4.8 or 6 mg N/g starch. In the case of urea, it is preferred to add from about 2.4 to about 12 mg urea per gram of starch, for example, 2.4, 4.8, 7.2, 9.6 or 12 mg urea per gram of starch. Yeast foods that supply, for example, vitamins (such a B vitamins and biotin), minerals (such as from salts of magnesium and zinc) and micronutrients and nutrients can be added to the fermentation medium. Yeast foods can include autolyzed yeast and plant extracts and are typically added to a concentration of from about 0.01 to about 1 g/L, for example from about 0.05 to about 0.5 g/L. Bactericides can also optionally be added to the fermentation medium. Examples of typical bactericides include virginiamycin, nisin, erythromycin, oleandomycin, fiavomycin, and penicillin G. In the case of virginiamycin, a concentration of from about 1 ppm to about 10 ppm is preferred.

In any of the various hexose and/or pentose sugar yeast fermentation aspects, the fermentation medium is typically inoculated with yeast to a concentration of between about 1×10⁸ cells/mL and about 1×10¹° cells/mL. Generally, yeast inoculum introduced into the fermentation vessel comprises the yeast dispersed throughout an aqueous medium. Typically, the yeast content of the yeast inoculum is from about 0.1 to about 5 wt. % and, more typically, from about 1 to about 2.5 wt. %. The relative proportions of yeast inoculum and cellulose hydrolyzate introduced into the fermentation vessel depend on a variety of factors including, for example, the composition of each stream. Generally, however, the mass ratio of yeast inoculum to hydrolyzate introduced into the fermentation vessel is from about 0.01:1 to about 0.25:1, or from about 0.02:1 to about 0.1:1. A fermentation temperature of from about 30° C. to about 36° C., from about 31° C. to about 35° C. or from about 32° C. to about 34° C. is preferred. Fermentation may occur for between about 45 hours and about 75 hours.

Hexose sugar fermenting organisms include yeasts. Any of a variety of yeasts can be employed as the yeast in the present process. Typical yeasts include any of a variety of commercially available yeasts, such as commercial strains of Saccharomyces cerevisiae. Suitable commercially available strains include ETHANOL RED (available from Red Star/Lesaffre, USA); BioFenn HP and XR (available from North American Bioproducts); FALI (available from Fleischmann's Yeast); SUPERSTART (available from Lallemand); GERT STRAND (available from Gert StrandAB, Sweden); FERMIOL (available from DSM Specialties); and Thennosac (available from Alltech). In some aspects, the hexose fermenting organism is a recombinant yeast having at least one transgene expressing an enzyme useful for converting mono- and/or oligo-saccharides to ethanol.

Suitable pentose sugar (e.g., xylose) fermenting organisms include yeasts. Such yeasts include Pachysolen tannophilus, Pichia stipites, Candida diddensii, Candida utilis, Candida tropicalis, Candida subtropicalis, Saccharomyces diastaticus, Saccharomycopsis fibuligera and Torula candida. In some aspects, the pentose fermenting organism is a recombinant yeast having at least one transgene expressing an enzyme useful for converting mono- and/or oligo-saccharides to ethanol. For instance, the genome of P. stipites may be incorporated into S. cerevisiae by a gene shuffling method to produce a hybrid yeast capable of producing bioethanol from xylose while retaining the ability to survive in high concentrations of ethanol.

In some aspects of the present invention, organisms capable of fermenting both hexose and pentose sugars are utilized to convert monosaccharides to ethanol. Typically, such organisms are strains of S. cerevisiae having transgenes encoding for one or more enzymes capable of converting pentose sugars to ethanol.

In some aspects of the present invention, the yeast may be adapted to the fermentation medium prior to fermentation to ethanol by propagating yeast in at least a portion of the fermentation medium. In some such aspects, a propagation mixture comprising the adapted yeast may optionally be initially charged to a fermentation vessel. Typically, such initial charge comprises about 2% to about 5% of the initial primary fermentation mixture volume. Propagation is typically performed by forming a propagation mixture comprising yeast, fermentation medium and additional nutrients. The propagation mixture may then be aerated. In aerobic conditions, the yeast preferentially converts glucose and other hexose sugars to form more yeast. It is believed that such yeast progeny are more efficient at converting hexose and pentose sugars to ethanol in an ethanol fermentation process performed on the fermentation medium. For batch propagation, propagation is performed for about 15 hours once all ingredients are added to the propagation vessel, after which time the contents of the propagation vessel are preferably transferred to a fermentation vessel.

At the end of fermentation, the alcohol content in the beer may range from about 3% to about 15% by weight as is basis, typically from 3% to about 10% or from about 5% to about 10% by weight as is basis, as measured by any suitable means, such as by high performance liquid chromatograph (HPLC), and corrected for suspended solids in the beer.

In aspects of the present invention wherein the fermentation medium comprises enzyme treated cellulosic biomass comprising cellulosic material such as cellulose, hemicellulose, lingo-cellulose, and glucan, the fermentation organism source may optionally comprise at least one species of cellulolytic organism capable of breaking down and metabolizing non-hydrolyzed cellulose, glucan or hemicellulose present in the fermentation medium to ethanol. Such cellulolytic organisms are known in the art and include Escherichia coli, Zymomonas mobilis, Bacillus stearothermophilus, Saccharomyces cerevisiae, Clostridia thermocellum, Thermoanaerobacterium saccharolyticum, Pichia stipites and Pachysolen tannophilus. Also within the scope of the present invention are cellulolytic bacteria having one or more transgenes encoding for the ethanol-producing pathway. In some other aspects of the present invention the fermentation organism source further comprises at least one species of cellulolytic organism capable of breaking down non-hydrolyzed hemicellulose present in the adjusted combined liquefaction admixture and synthesizing ethanol.

Generally, the beer is a mixture of water, ethanol, unconverted hexose and pentose sugars, fibers (e.g., cellulose, hemicellulose, lingo-cellulose and lignin) and ash. The overall composition of the beer generally varies depending on, for example, the composition of the enzymatic hydrolyzate, the ethanol fermentation organism source, and the relative proportions introduced into the saccharification and fermentation vessels. Preferably, the composition of the fermentate represents suitable yields of ethanol based on the fermentable sugar content of the fermentation medium. For example, generally the process of the present invention provides for an ethanol yield of 50%, 60%, 70% or 80%, and ranges thereof, based on the total carbohydrate contained in the solid waste.

### Fermentation Product Recovery

Fermentation products may be recovered using any of various methods known in the art. For instance, fermentation products may be separated from other fermentation components by distillation (e.g., azeotropic distillation), liquid-liquid extraction, solid-liquid extraction, adsorption, gas stripping, membrane evaporation, pervaporation, centrifugation, crystallization, filtration, microfiltration, nanofiltration, ion exchange, or electrodialysis. As a specific example, methanol or other fermentation products having sufficient volatility may be recovered from a fermentation mixture by distillation. In another example, 1-butanol may be isolated from a fermentation mixture using methods known in the art for acetone-butanol-ethanol (“ABE”) fermentations (see for example, Durre, Appl. Microbiol. Biotechnol. 49:639-648 (1998), Groot et al., Process. Biochem. 27:61-75 (1992), and references therein), for instance by solids removal followed by isolation by distillation, liquid-liquid extraction, adsorption, gas stripping, membrane evaporation, or pervaporation. In yet another example, 1,3-propanediol may be isolated from a fermentation mixture by extraction with an organic solvent, distillation, and column chromatography (see U.S. Pat. No. 5,356,812). In yet another example, amino acids may be collected from fermentation mixture by methods such as ion-exchange resin adsorption and/or crystallization. Selection of a suitable separation method for any particular fermentation product may be done by those skilled in the art.

In one aspect of the present invention, depicted in FIGS. 1 and 2, after the ethanolic fermentation is complete, the beer 93 is fed to a distillation system 100 comprising a reboiler where ethanol 101 and volatile impurities (e.g., fusel oil (predominantly comprising amyl alcohol), acetic acid and furfural) are separated by vaporization in a distillation column leaving liquid reboiler bottoms (stillage) 102 containing dissolved solids. Generally, conventional distillation apparatuses known in the art are suitable for use in accordance with the present invention. Conventional apparatuses are described, for instance, in Distillation Technology, GEA Wiegand GmbH, publication P06E 022009 (2013) and Bioethanol Technology, GEA Wiegand GmbH, publication P11E (2013), the entire contents of which are incorporated herein by reference for all relevant purposes. Examples of suitable distillation columns include columns having dual flow and cross flow trays, such as dual flow sieve trays or cross-flow valve trays. In some aspects, cross flow valve trays are used because of the higher tum down ratio and higher they provide. Suitable valve trays include, for example, NORPRO PROVALVE trays. Ethanol is condensed and purified in the distillation column. The liquid ethanol exits the top of the distillation column at about 95% purity from where it passes through a molecular sieve dehydration column which removes at least about 75%, about 80%, about 85%, about 90%, about 95% or even about 99% of the remaining residual water. In further reference to FIGS. 1 and 2, in one aspect of the present invention, the liquid reboiler bottoms (i.e., still bottoms) 102 are fed to a solid-liquid separation device 110, such as a filter press or centrifuge, in order to separate the solids 112 from the liquid 111 (termed “thin stillage”). The still bottoms may be optionally be incinerated for heat recovery, composted or used as a soil amendment. The thin stillage may be recycled to any of the pulper, heavy/coarse contaminant washing, crude cellulosic fiber washing, acid impregnation or enzyme hydrolysis. In some aspects, at least a portion of the still bottom solids may be recycled to the pulper or acid impregnation. In some other aspects of the present invention, the still bottom solids can be dried and used as an animal feed supplement.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A process for producing a monosaccharide composition from solid waste, the process comprising:
(i) forming a solid waste slurry comprising solid waste and water, wherein the slurry comprises from about 2 to about 8 percent by weight total solids, and wherein the solid waste comprises cellulose and ash;
(ii) pulping the solid waste slurry to form a pulped solid waste slurry comprising cellulosic fiber;
(iii) (a) fractionating the pulped solid waste slurry through a filtration medium having opening of from about 0.2 cm to about 2 cm to form an oversize stream comprising coarse contaminants and an undersize stream comprising cellulosic fibers and light contaminants and (b) fractionating the undersize stream to form a light contaminant stream comprising ash and a cellulosic fiber stream, wherein the cellulosic fiber stream comprises no more than about 15 percent by ash on a dry basis, and wherein the ash content of the cellulosic fiber stream is less than the ash content of the solid waste on a dry basis;
(iv) dewatering the cellulosic fiber stream to form a dewatered cellulosic fiber stream having a solids content of from about 25 wt. % to about 60 wt. % and a dewatered cellulosic fiber aqueous stream;
(v) treating the dewatered cellulosic fiber stream by contact with steam at a treatment temperature of at least 150° C. and a treatment pressure of at least 350 kPa (about 50 psig) to form steam treated cellulosic fiber; and
(vi) forming a slurry comprising the steam treated cellulosic fiber, water and at least one cellulase enzyme, and incubating the slurry to form a hydrolyzed cellulosic composition comprising soluble glucose.

2. The process of claim 1 wherein the solid waste slurry pH is from about 3 to about 6.

3. The process of claim 1 wherein the solid waste slurry pH is from about 4 to about 8.

4. The process of claim 1 wherein the pulped solid waste slurry is diluted with process make-up water, recycled wash water, condensate, or a combinations thereof, to a concentration of from about 1 wt. % to about 4 wt. % total solids prior to fractionation.

5. The process of claim 1 wherein the light contaminant stream is diluted to a solids concentration of from about 1 wt. % to about 2 wt. %, the diluted light contaminant stream is fractionated to from an oversize stream comprising light contaminants and an undersize stream comprising cellulosic fiber, the overhead stream is washed with make-up process water, recycled wash water, condensate, or combinations thereof, to form a wash stream comprising cellulosic fiber, and wherein the wash stream and the undersize stream are recycled to the solid waste slurry.

6. The process of claim 1 wherein the pulped solid waste slurry further comprises a heavy contaminant fraction, the process further comprising processing the pulped solid waste slurry in a high density cleaner to remove at least a portion of the heavy contaminants fraction prior to fractionating the pulped solid waste slurry.

7. The process of claim 1 further comprising thickening the cellulosic fiber stream prior to dewatering to increasing the total solid concentration in the cellulosic fiber stream to from about 8 wt. % to about 12 wt. % by removal of an aqueous stream therefrom.

8. The process of claim 1 wherein the dewatered cellulosic fiber stream comprises from about 25 wt. % to about 45 wt. % total solids.

9. The process of claim 1 wherein the cellulosic fiber aqueous stream comprises sediment, wherein at least a portion of the sediment is removed from said aqueous stream, and wherein at least about 25% of the resulting aqueous stream is recycled to the pulper.

10. The process of claim 1 further comprising milling the dewatered cellulosic fiber stream prior to treating with steam, wherein a plurality of the milled cellulosic fibers are characterized as having an average fiber length of less than about 500 microns.

11. The process of claim 1 further comprising (i) treating the dewatered cellulosic fiber stream with acid in an acid impregnation vessel prior to treatment with steam wherein the dewatered cellulosic fiber stream is combined with a mineral acid having an acid concentration of from about 0.5 wt. % to about 4 wt. % acid in the acid impregnation vessel to form an acid impregnation slurry comprising cellulosic fiber, the slurry having a total solids content of from about 3 wt. % to about 7 wt. %, (ii) dewatering the acid impregnation slurry to form an acid impregnation liquid stream and acid impregnated cellulosic fiber having a total solids content of from about 35 wt. % to about 65 wt. %, wherein the acid impregnated cellulosic fiber has an acid concentration of from about 0.01 to about 0.05 kg acid per kg of cellulose on a dry weight basis.

12. The process of claim 11 further comprising adding supplemental cellulosic waste to the acid impregnation vessel, the supplemental waste comprising food waste, lawn and garden waste, wood, agricultural residues, and combinations thereof.

13. The process of claim 1 further comprising (i) admixing the dewatered cellulosic fiber stream with acid in an acid impregnation mixer prior to treatment with steam, the acid impregnation mixer having a mineral acid source, wherein the dewatered cellulosic fiber stream is admixed with a mineral acid having a concentration of from 0.5 wt. % to about 4 wt. % to produce an acid impregnated cellulosic fiber having a total solids content of from about 20 wt. % to about 55 wt. %, and having an acid concentration of from about 0.01 to about 0.05 kg acid per kg of dewatered cellulosic fiber stream on a dry weight basis and (ii) transferring the acid impregnated cellulosic fiber to a hold vessel and maintaining the acid impregnated cellulosic fiber in the hold vessel for an average residence time of from about 5 to about 60 minutes at a temperature of from about 30° C. to about 70° C.

14. The process of claim 1 wherein steam treatment comprises a first treatment stage and a second treatment stage, wherein:
(i) the first treatment stage is conducted at a treatment temperature of at least about 150° C. and a treatment gauge pressure of at least about 350 kPa and the steam addition is direct addition; and
(ii) the second treatment stage is conducted at a pressure differential of least about 35 kPa less than the first treatment stage pressure, and the pressure in the second treatment stage is controlled above atmospheric pressure.

15. The process of claim 14 wherein the first treatment stage produces a volatilized fraction of the steam treated feedstock, the volatized fraction comprising volatile organic compounds, wherein at least a portion of the volatilized fraction is continuously or discontinuously released from the first treatment stage.

16. The process of claim 14 wherein the second treatment stage produces a volatilized fraction of the steam treated feedstock, the volatized fraction comprising volatile organic compounds, wherein at least a portion of the volatilized fraction is continuously or discontinuously released from the second treatment stage.

17. The process of claim 1 wherein the total yield of glucose in the hydrolyzed cellulosic composition is from about 30% to about 80% based on the total cellulose content of the slurry comprising steam treated cellulosic fiber.

18. The process of claim 1 wherein the total yield of glucose in the hydrolyzed cellulosic composition is at least about 40% of theoretical value.

19. The process of claim 1 wherein the slurry comprising steam treated cellulosic fiber and cellulase enzyme further comprises at least one hemicellulase enzyme.

20. The process claim 1 further comprising inoculating the hydrolyzed cellulosic composition with a source of at least one microorganism capable of converting glucose to a fermentation product and incubating the composition to form a fermentation mixture comprising the fermentation product.

21. The process of claim 20 wherein the microorganism is selected from native or recombinant yeast, bacteria, filamentous fungi, microalgae, and combinations thereof.

22. The process of claim 20 wherein the source of microorganism comprises Saccharomyces cerevisiae and the fermentation product is ethanol.

23. The process of claim 20 wherein the hydrolyzed cellulosic composition further comprises soluble pentose monosaccharide and the source of microorganism (i) further comprises an organism capable of converting pentose monosaccharide to ethanol or (ii) comprises an organism capable of converting both hexose monosaccharide and pentose monosaccharide to ethanol.
